(19) United States
(12) Patent Application Publication
(10) Pub. No.: US 2022/0061635 A1
(43) Pub. Date: Mar. 3, 2022

(54) ENDOSCOPE DEVICE, METHOD OF OPERATING ENDOSCOPE DEVICE, AND RECORDING MEDIUM

(71) Applicant: OLYMPUS CORPORATION, Tokyo (JP)

(72) Inventors: Masanori MAKI, Hachioji-shi (JP); Hideaki TAKAHASHI, Musashimurayama-shi (JP)

(73) Assignee: OLYMPUS CORPORATION, Tokyo (JP)

(21) Appl. No.: 17/341,503

(22) Filed: Jun. 8, 2021

(30) Foreign Application Priority Data
Aug. 26, 2020 (JP) 2020-142645

(51) Int. Cl. A61B 1/00

(52) U.S. Cl. CPC A61B 1/00096; A61B 2562/0219; A61B 1/00147; A61B 1/00013

(57) ABSTRACT

An endoscope device includes an endoscope including, in a tip end thereof, an imaging device, a first optical system, and an optical element. The optical element is disposed at any one of a first position on an optical path of the first optical system and a second position away from the optical path of the first optical system and is configured to switch between states of light incident on the imaging device. In addition, the endoscope device includes a detector and a processor. The detector is configured to detect movement of the optical element. The processor is configured to determine whether or not the optical element moves from the first position or the second position on the basis of the movement. The processor is configured to control a position of the optical element on the basis of a result of determination executed by the processor.

### BACKGROUND OF THE INVENTION

### Field of the Invention

The present invention relates to an endoscope device, a method of operating an endoscope device, and a recording medium.

Priority is claimed on Japanese Patent Application No. 2020-142645, filed on Aug. 26, 2020, the content of which is incorporated herein by reference.

### Description of Related Art

Industrial endoscopes are used for visual inspection for scratches, corrosion, or the like in a concealed place such as the inside of engines, turbines, chemical plants, and the like. Countermeasures such as repairs vary in accordance with the degree of defects such as scratches or corrosion. Therefore, a measurement function of measuring the size of scratches, corrosion, or the like is important.

An exemplary measurement method is disclosed in Japanese Unexamined Patent Application, First Publication No. 2004-049638. A device using this method includes two optical systems having parallax with each other. The two optical systems simultaneously generate two optical images, and an imaging device (image sensor) simultaneously generates two images on the basis of the two optical images. The device calculates three-dimensional coordinates of a subject and the size of the subject on the basis of the principle of stereo measurement by using the two images corresponding to the two optical images.

The device shown in Japanese Unexamined Patent Application, First Publication No. 2004-049638 includes two optical systems, and light passes through two different paths. Two optical images of a subject are formed in two different regions on one imaging device. Since one optical image is formed in only half a region of the imaging device and a field angle narrows, there is a shortcoming in that observation performance deteriorates.

In a case in which two imaging devices are used, a field angle expands and the above-described shortcoming is resolved. However, miniaturization of imaging devices is difficult at present. In order to resolve the above-described shortcoming in endoscopes of which miniaturization is important, an optical device is disclosed in Japanese Unexamined Patent Application, First Publication No. 2010-128354. This optical device has two optical paths and switches between the two optical paths by using an optical path-switching mechanism. In this way, an optical image of light passing through each of the two optical paths is formed in the entire region on the imaging device. Switching between optical paths is necessary in order to acquire two images having parallax with each other. Therefore, a time difference occurs between imaging timings of the two images. However, observation with a wider field angle is available compared to the device shown in Japanese Unexamined Patent Application, First Publication No. 2004-049638 since an optical image of light passing through each optical system is formed in the entire region on the imaging device.

### SUMMARY OF THE INVENTION

According to a first aspect of the present invention, an endoscope device includes an endoscope, a detector, and a processor. The endoscope includes, in a tip end thereof, an imaging device, a first optical system, and an optical element. The first optical system is configured to lead light from a subject to the imaging device. The optical element is disposed at any one of a first position on an optical path of the first optical system and a second position away from the optical path of the first optical system and is configured to switch between states of light incident on the imaging device. The detector is configured to detect movement of the optical element. The processor is configured to determine whether or not the optical element moves from the first position or the second position on the basis of the movement. The processor is configured to execute disposition control of disposing the optical element at the first position or the second position. The processor is configured to execute the disposition control in a predetermined state. The predetermined state is a state in which the processor determines that the optical element moves from the first position or the second position after the disposition control is executed and before the disposition control is executed next.

According to a second aspect of the present invention, in the first aspect, the processor may be configured to determine whether or not the optical element moves from the first position or the second position by determining whether or not a force exceeding a predetermined amount is added to the optical element.

According to a third aspect of the present invention, in the first aspect, the processor may be configured to determine that the optical element moves from the first position or the second position when relative movement of the optical element to the imaging device occurs.

According to a fourth aspect of the present invention, in the first aspect, the endoscope may include a second optical system that is different from the first optical system and is configured to lead light from the subject to the imaging device. The optical element may be a shutter that cuts off light. The second position may be on an optical path of the second optical system. The imaging device may be configured to generate a first image on the basis of a first optical image formed by light passing through the first optical system when the optical element is disposed at the second position. The imaging device may be configured to generate a second image on the basis of a second optical image formed by light passing through the second optical system when the optical element is disposed at the first position. The imaging device may be configured to execute stereo imaging for generating the first image one or more times and generating the second image one or more times.

According to a fifth aspect of the present invention, in the fourth aspect, the processor may be configured to dispose the optical element at an initial position that is set in advance among the first position and the second position by executing the disposition control and causing the imaging device to start the stereo imaging. After the imaging device starts the stereo imaging, the processor may be configured to execute the disposition control of disposing the optical element at any one of the first position and the second position in the predetermined state and cause the imaging device to continue the stereo imaging

According to a sixth aspect of the present invention, in the fifth aspect, after the imaging device starts the stereo imaging, the processor may be configured to execute the disposition control of disposing the optical element at the initial position in the predetermined state so as to cause the imaging device to continue the stereo imaging.

According to a seventh aspect of the present invention, in the fourth aspect, the processor may be configured to confirm a position of the optical element in the predetermined state. The processor may be configured to skip executing the disposition control and cause the imaging device to continue the stereo imaging when the confirmed position is the same as a predetermined position necessary for the imaging device to next generate the first image or the second image. The processor may be configured to execute the disposition control and cause the imaging device to continue the stereo imaging when the confirmed position is different from the predetermined position. The predetermined position is any one of the first position and the second position.

According to an eighth aspect of the present invention, in the first aspect, in the predetermined state, by executing the disposition control, the processor may be configured to dispose the optical element at any one of an initial position that is set in advance among the first position and the second position, a position at which the optical element is disposed through the disposition control executed immediately before the predetermined state occurs, and a position at which the optical element is scheduled to be disposed through the disposition control to be executed next.

According to a ninth aspect of the present invention, in the first aspect, the optical element may be a lens. The processor may be configured to dispose the optical element at any one of the first position and the second position so as to control a focus state of light incident on the imaging device.

According to a tenth aspect of the present invention, in the first aspect, the endoscope device may further include a magnetic actuator configured to move the optical element from the first position to the second position or from the second position to the first position. The detector may be configured to detect the movement by detecting a current generated in the magnetic actuator.

According to an eleventh aspect of the present invention, in the first aspect, the detector may be an acceleration sensor or a gyro sensor.

According to a twelfth aspect of the present invention, in the first aspect, the detector may be configured to detect the movement on the basis of an image generated by the imaging device.

According to a thirteenth aspect of the present invention, in the second aspect, the endoscope device may further include a holding mechanism configured to hold the optical element at the first position or the second position by using a predetermined amount of force.

According to a fourteenth aspect of the present invention, a method of operating an endoscope device is provided. The endoscope includes, in a tip end thereof, an imaging device, a first optical system, and an optical element. The first optical system is configured to lead light from a subject to the imaging device. The optical element is disposed at any one of a first position on an optical path of the first optical system and a second position away from the optical path of the first optical system and is configured to switch between states of light incident on the imaging device. The method includes a detection step, a determination step, a first control step, and a second control step. The method causes a detector to detect movement of the optical element in the detection step. The method causes a processor to determine whether or not the optical element moves from the first position or the second position on the basis of the movement in the determination step. The method causes the processor to execute disposition control of disposing the optical element at the first position or the second position in the first control step. The method causes the processor to execute the disposition control in a predetermined state in the second control step. The predetermined state is a state in which the processor determines that the optical element moves from the first position or the second position after the disposition control is executed and before the disposition control is executed next.

According to a fifteenth aspect of the present invention, a non-transitory computer-readable recording medium saves a program executed by a computer of an endoscope device is provided. The endoscope includes, in a tip end thereof, an imaging device, a first optical system, and an optical element. The first optical system is configured to lead light from a subject to the imaging device. The optical element is disposed at any one of a first position on an optical path of the first optical system and a second position away from the optical path of the first optical system and is configured to switch between states of light incident on the imaging device. The computer executes a detection step, a determination step, a first control step, and a second control step. The computer detects movement of the optical element in the detection step. The computer determines whether or not the optical element moves from the first position or the second position on the basis of the movement in the determination step. The computer executes disposition control of disposing the optical element at the first position or the second position in the first control step. The computer executes the disposition control in a predetermined state in the second control step. The predetermined state is a state in which the processor determines that the optical element moves from the first position or the second position after the disposition control is executed and before the disposition control is executed next.

### BRIEF DESCRIPTION OF THE DRAWINGS

FIG. 1 is a block diagram showing a configuration of an endoscope device according to a first embodiment of the present invention.

FIG. 2 is a flow chart showing a procedure of an operation of the endoscope device according to the first embodiment of the present invention.

FIG. 3 is a block diagram showing a configuration of an endoscope device according to a modified example of the first embodiment of the present invention.

FIG. 4 is a block diagram showing a configuration of the endoscope device according to the modified example of the first embodiment of the present invention.

FIG. 5 is a block diagram showing a configuration of an endoscope device according to a second embodiment of the present invention.

FIG. 6 is a block diagram showing a configuration of the endoscope device according to the second embodiment of the present invention.

FIG. 7 is a perspective view showing a configuration of a magnetic actuator included in the endoscope device according to the second embodiment of the present invention.

FIG. 8 is a perspective view showing a configuration of the magnetic actuator included in the endoscope device according to the second embodiment of the present invention.

FIG. 9 is a graph showing a waveform of a digital signal input into a determination unit included in the endoscope device according to the second embodiment of the present invention.

FIG. 10 is a graph showing the gravitational acceleration extracted from the digital signal in the second embodiment of the present invention.

FIG. 11 is a graph showing an acceleration obtained by eliminating the gravitational acceleration and noise from the digital signal in the second embodiment of the present invention.

FIG. 12 is a flow chart showing a procedure of processing executed by the endoscope device according to the second embodiment of the present invention.

FIG. 13 is a flow chart showing a procedure of processing executed by the endoscope device according to the second embodiment of the present invention.

FIG. 14 is a timing chart showing an image-generation sequence in an endoscope device compared with the endoscope device according to the second embodiment of the present invention.

FIG. 15 is a timing chart showing an image-generation sequence in the endoscope device according to the second embodiment of the present invention.

FIG. 16 is a timing chart showing an image-generation sequence in the endoscope device according to the second embodiment of the present invention.

FIG. 17 is a timing chart showing an image-generation sequence in an endoscope device compared with the endoscope device according to the second embodiment of the present invention.

FIG. 18 is a timing chart showing an image-generation sequence in the endoscope device according to the second embodiment of the present invention.

FIG. 19 is a block diagram showing a configuration of an endoscope device according to a third embodiment of the present invention.

FIG. 20 is a block diagram showing a configuration of an endoscope device according to a fourth embodiment of the present invention.

FIG. 21 is a perspective view showing a configuration of a magnetic actuator included in the endoscope device according to the fourth embodiment of the present invention.

FIG. 22 is a block diagram showing a configuration of an endoscope device according to a fifth embodiment of the present invention.

FIG. 23 is a block diagram showing a configuration of the endoscope device according to the fifth embodiment of the present invention.

FIG. 24 is a block diagram showing a configuration of an endoscope device according to a sixth embodiment of the present invention.

FIG. 25 is a timing chart showing an exposure period in an imaging device using a global shutter method.

FIG. 26 is a timing chart showing an exposure period in an imaging device using a rolling shutter method.

FIG. 27 is a block diagram showing a configuration of an endoscope device according to a seventh embodiment of the present invention.

FIG. 28 is a perspective view showing a configuration of a magnetic actuator included in the endoscope device according to the seventh embodiment of the present invention.

FIG. 29 is a perspective view showing a configuration of the magnetic actuator included in the endoscope device according to the seventh embodiment of the present invention.

FIG. 30 is a flow chart showing a procedure of processing executed by the endoscope device according to the seventh embodiment of the present invention.

FIG. 31 is a timing chart showing an image-generation sequence in the endoscope device according to the seventh embodiment of the present invention.

FIG. 32 is a timing chart showing an image-generation sequence in the endoscope device according to the seventh embodiment of the present invention.

FIG. 33 is a timing chart showing an image-generation sequence in the endoscope device according to the seventh embodiment of the present invention.

FIG. 34 is a timing chart showing an image-generation sequence in the endoscope device according to the seventh embodiment of the present invention.

FIG. 35 is a block diagram showing a configuration of an endoscope device according to an eighth embodiment of the present invention.

### DETAILED DESCRIPTION OF THE INVENTION

Hereinafter, embodiments of the present invention will be described with reference to the drawings.

### First Embodiment

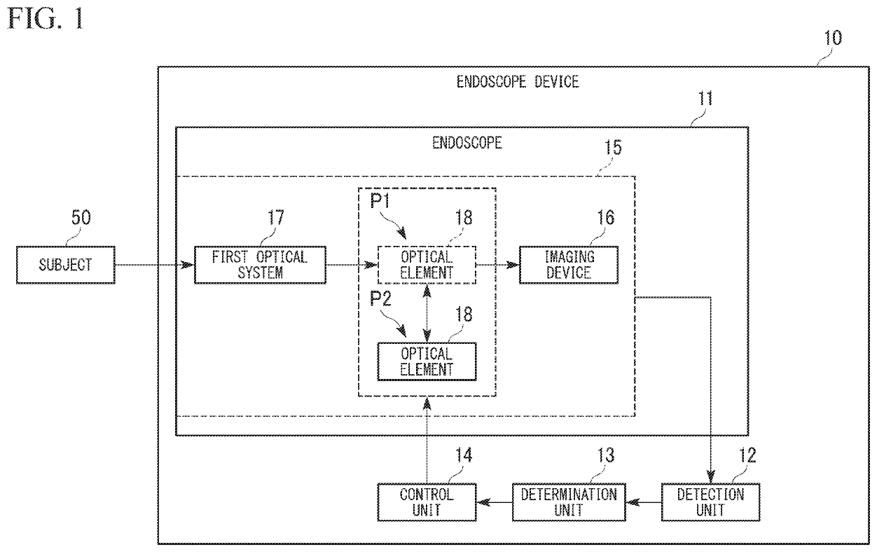

FIG. 1 shows a configuration of an endoscope device 10 according to a first embodiment of the present invention. The endoscope device 10 shown in FIG. 1 includes an endoscope 11, a detection unit 12, a determination unit 13, and a control unit 14. The endoscope 11 includes an imaging device 16, a first optical system 17, and an optical element 18. The imaging device 16, the first optical system 17, and the optical element 18 are disposed in a distal end 15 of the endoscope 11.

The first optical system 17 leads light from a subject 50 to the imaging device 16. The optical element 18 is disposed at any one of a first position P1 on an optical path of the first optical system 17 and a second position P2 away from the optical path of the first optical system 17 and switches between states of light incident on the imaging device 16. The detection unit 12 detects movement of the optical element 18. The determination unit 13 determines whether or not the optical element 18 moves from the first position P1 or the second position P2 on the basis of the movement of the optical element 18. The control unit 14 executes disposition control of disposing the optical element 18 at the first position P1 or the second position P2. In addition, the control unit 14 executes the disposition control in a predetermined state again. The predetermined state is a state in which the determination unit 13 determines that the optical element 18 moves from the first position P1 or the second position P2 after the disposition control is executed and before the disposition control is executed next.

For example, the optical element 18 switches between a first state and a second state. For example, light passing through the first optical system 17 is incident on the imaging device 16 in the first state and is not incident on the imaging device 16 in the second state. Alternatively, light passing through only the first optical system 17 is incident on the imaging device 16 in the first state, and the light passing through the first optical system 17 and the optical element 18 is incident on the imaging device 16 in the second state. The control unit 14 disposes the optical element 18 at one of the first position P1 and the second position P2 by executing the disposition control.

In the example shown in FIG. 1, the optical element 18 is disposed between the first optical system 17 and the imaging device 16. The first optical system 17 may be disposed between the optical element 18 and the imaging device 16. The endoscope 11 may include at least one of the detection unit 12, the determination unit 13, and the control unit 14. At least one of the detection unit 12, the determination unit 13, and the control unit 14 may be disposed in the distal end 15.

For example, the detection unit 12 is constituted by a sensor or a processor (control circuit). For example, the determination unit 13 and the control unit 14 are constituted by a processor. In a case in which the detection unit 12 is constituted by a processor, the determination unit 13 and the control unit 14 are constituted by a processor the same as or different from the processor constituting the detection unit 12. The determination unit 13 and the control unit 14 may be constituted by processors different from each other.

For example, the optical element 18 is a shutter or a lens. The optical element 18 is able to move between the first position P1 and the second position P2. The optical element 18 is able to move from the first position P1 to the second position P2 or from the second position P2 to the first position P1.

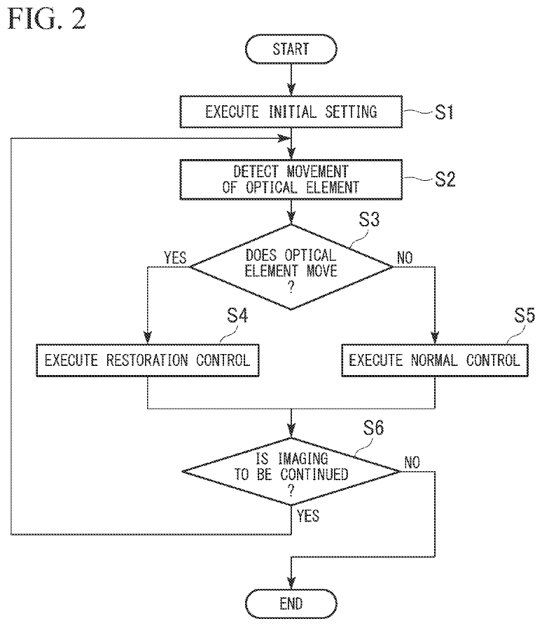

FIG. 2 shows a procedure of an operation of the endoscope device 10. The operation of the endoscope device 10 will be described with reference to FIG. 2.

First, initial setting is executed. At this time, sequence information indicating details of an image-generation sequence for generating images is set in the endoscope device 10. For example, the sequence information indicates the number of times of imaging executed by the imaging device 16 and indicates a position of the optical element 18 at each time of imaging. One or more images are generated in the image-generation sequence. The control unit 14 executes the disposition control and moves the optical element 18 to the position indicated by the sequence information (Step S1).

After Step S1, the detection unit 12 detects movement of the optical element 18. For example, the detection unit 12 detects absolute movement of the optical element 18 or relative movement of the optical element 18 to the imaging device 16 (Step S2).

After Step S2, the determination unit 13 determines whether or not the optical element 18 moves from the first position P1 or the second position P2 on the basis of the movement of the optical element 18 (Step S3). The determination unit 13 determines whether or not the optical element 18 moves due to a different cause from the disposition control executed by the control unit 14. When the optical element 18 actually moves or it is estimated that the optical element 18 moves, the determination unit 13 determines that the optical element 18 moves.

When the determination unit 13 determines that the optical element 18 moves from the first position P1 or the second position P2 in Step S3, the control unit 14 executes restoration control (Step S4).

For example, after the optical element 18 is disposed at the second position P2, there is a possibility that the determination unit 13 determines that the optical element 18 moves. In such a case, there is a possibility that the optical element 18 has moved from the second position P2 to the first position P1. Therefore, the control unit 14 executes control of moving the optical element 18 to the second position P2 in Step S4.

After the optical element 18 moves to a correct position, imaging may be resumed in order to execute the rest of the image-generation sequence. After the optical element 18 moves to a correct position, imaging may be started from the beginning of the image-generation sequence.

When the determination unit 13 determines that the optical element 18 does not move in Step S3, normal control is executed (Step S5). For example, the imaging device 16 executes imaging and generates an image in Step S5.

After Step S4 or Step S5, it is determined whether or not to continue imaging (Step S6). For example, if the imaging device 16 has not executed imaging by the number of times indicated by the sequence information, it is determined to continue imaging. If the imaging device 16 has executed imaging by the number of times indicated by the sequence information, it is determined not to continue imaging.

When it is determined to continue imaging in Step S6, Step S2 is executed. When it is determined not to continue imaging in Step S6, the processing shown in FIG. 2 is completed.

In the first embodiment, the control unit 14 controls the position of the optical element 18 on the basis of the result that the determination unit 13 determines movement of the optical element 18. Therefore, the endoscope device 10 can suppress the occurrence of a situation in which the imaging device 16 generates an image with the optical element 18 being disposed at an incorrect position.

### Modified Example of First Embodiment

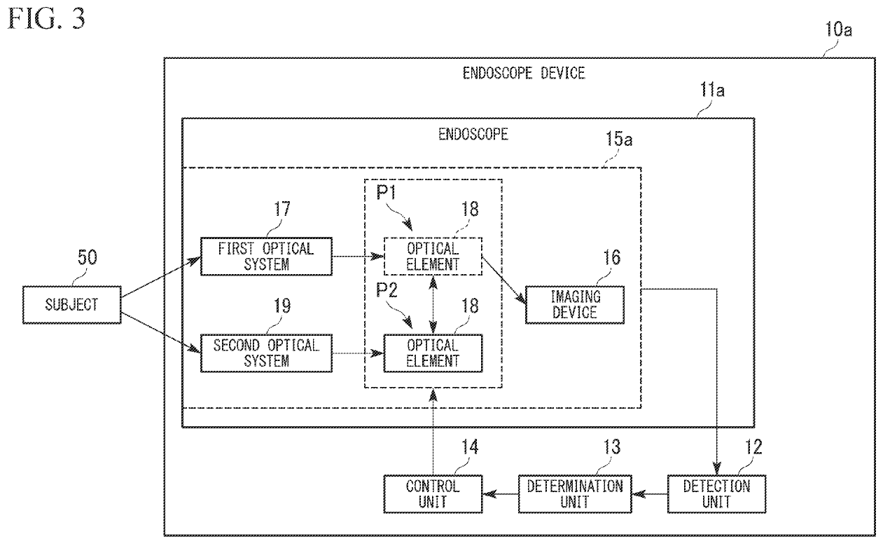

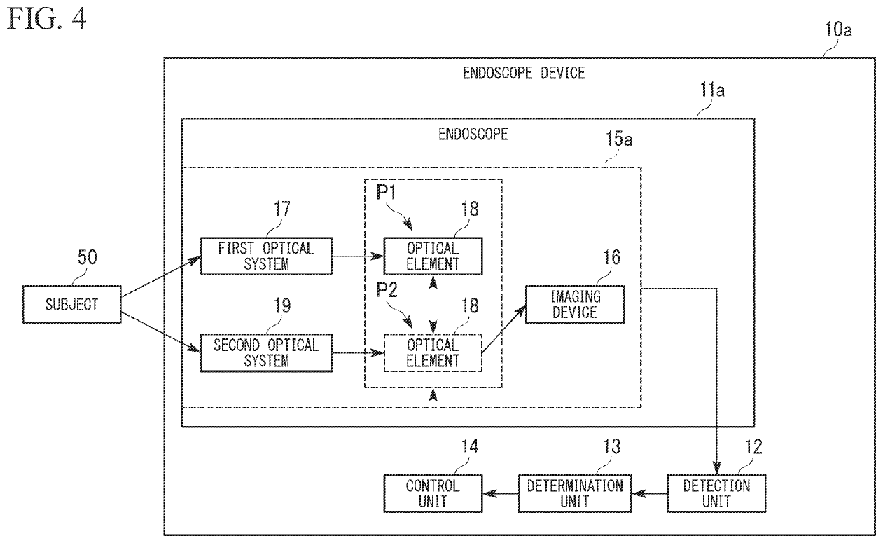

FIG. 3 and FIG. 4 show a configuration of an endoscope device 10a according to a modified example of the first embodiment of the present invention. The endoscope device 10a shown in FIG. 3 and FIG. 4 includes an endoscope 11a, a detection unit 12, a determination unit 13, and a control unit 14. The endoscope 11a includes an imaging device 16, a first optical system 17, an optical element 18, and a second optical system 19. The imaging device 16, the first optical system 17, the optical element 18, and the second optical system 19 are disposed in a distal end 15a of the endoscope 11a. The same configuration as that shown in FIG. 1 will not be described.

The second optical system 19 leads light from a subject 50 to the imaging device 16. The optical element 18 is a shutter that cuts off light. The second position P2 is on an optical path of the second optical system 19. When the optical element 18 is disposed at the second position P2, the imaging device 16 generates a first image on the basis of a first optical image formed by light passing through the first optical system 17. When the optical element 18 is disposed at the first position Pl, the imaging device 16 generates a second image on the basis of a second optical image formed by light passing through the second optical system 19. The first optical image and the second optical image include an optical image (subject image) of the subject 50. The imaging device 16 executes stereo imaging for generating the first image one or more times and generating the second image one or more times.

For example, the optical element 18 switches between a first state and a second state. For example, in the first state, light passing through the first optical system 17 is incident on the imaging device 16, and light passing through the second optical system 19 is not incident on the imaging device 16. In the second state, light passing through the second optical system 19 is incident on the imaging device 16, and light passing through the first optical system 17 is not incident on the imaging device 16.

FIG. 3 shows an example in which the optical element 18 is disposed at the second position P2. Light emitted from the subject 50 passes through the first optical system 17. On the other hand, light emitted from the subject 50 passes through the second optical system 19. The light passing through the first optical system 17 is not blocked by the optical element 18 and is incident on the imaging device 16. The light forms the first optical image on the imaging device 16. The light passing through the second optical system 19 is blocked by the optical element 18 and is not incident on the imaging device 16. The imaging device 16 generates the first image on the basis of the first optical image.

FIG. 4 shows an example in which the optical element 18 is disposed at the first position P1. The light passing through the first optical system 17 is blocked by the optical element 18 and is not incident on the imaging device 16. The light passing through the second optical system 19 is not blocked by the optical element 18 and is incident on the imaging device 16. The light forms the second optical image on the imaging device 16. The imaging device 16 generates the second image on the basis of the second optical image.

In the modified example of the first embodiment, the endoscope device 10a can suppress the occurrence of a situation in which the imaging device 16 executes stereo imaging with the optical element 18 being disposed at an incorrect position.

### Second Embodiment

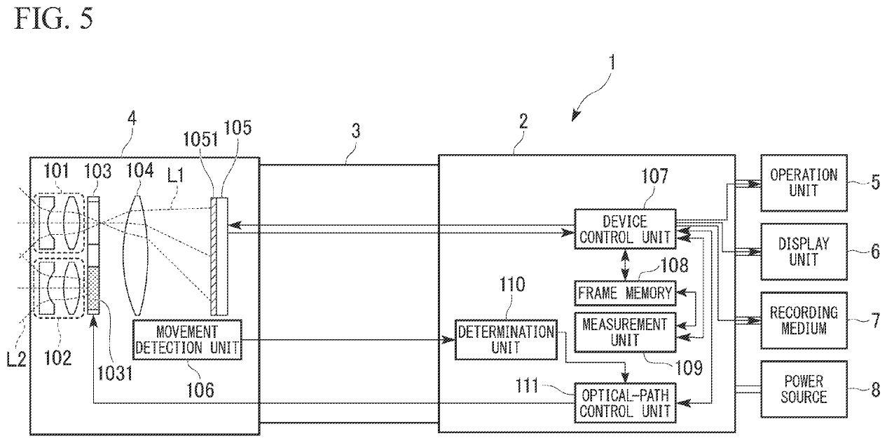

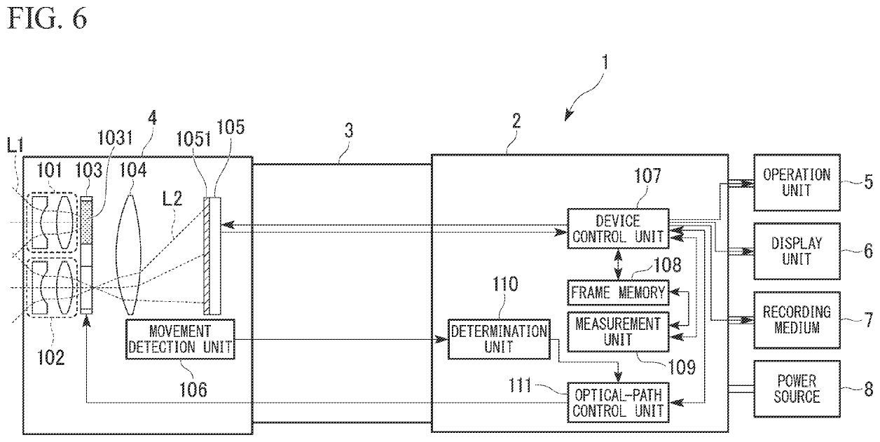

FIG. 5 and FIG. 6 show a configuration of an endoscope device 1 according to a second embodiment of the present invention. The endoscope device 1 shown in FIG. 5 and FIG. 6 includes a main body unit 2, an insertion unit 3, a distal end part 4, an operation unit 5, a display unit 6, a recording medium 7, and a power source 8. The insertion unit 3 is to be inserted into the inside of an object that is a target for measurement, and the distal end part 4 is disposed in the distal end of the insertion unit 3. The insertion unit 3 and the distal end part 4 are an example of the endoscope 11 shown in FIG. 1. The operation unit 5, the display unit 6, the recording medium 7, and the power source 8 are connected to the main body unit 2.

The distal end part 4 includes a first optical system 101, a second optical system 102, an optical path-switching unit 103, an imaging optical system 104, an imaging device 105, and a movement detection unit 106. The distal end part 4 may be attachable to and detachable from the insertion unit 3. The distal end part 4 may be always fixed to the insertion unit 3. The distal end part 4 is an example of the distal end 15 shown in FIG. 1.

The first optical system 101 forms a first optical path L1. The first optical system 101 is an example of the first optical system 17 shown in FIG. 1. The second optical system 102 forms a second optical path L2. The second optical system 102 is an example of the second optical system 19 shown in FIG. 3 and FIG. 4.

Each of the first optical system 101 and the second optical system 102 includes an objective lens constituted by a combination of a concave lens and a convex lens. The second optical system 102 is disposed so as to have parallax with the first optical system 101. In other words, the first optical system 101 and the second optical system 102 are disposed away from each other in a parallax direction. The parallax direction is a direction of a straight line passing through the optical center (principal point) of the first optical system 101 and the optical center (principal point) of the second optical system 102. The parallax direction is almost orthogonal to the optical axis of each optical system. Light incident on the first optical system 101 passes through the first optical path L1. Light incident on the second optical system 102 passes through the second optical path L2 different from the first optical path L1. The first optical system 101 forms a first optical image of a subject, and the second optical system 102 forms a second optical image of the subject.

The imaging optical system 104 forms, in an imaging region 1051 of the imaging device 105, an optical image of the subject formed by any one of the light passing through the first optical path L1 and the light passing through the second optical path L2. In other words, the imaging optical system 104 forms, in the imaging region 1051 of the imaging device 105, an optical image on the basis of light passing through an optical path that has been set as an optical path for imaging among the first optical path L1 and the second optical path L2. Hereinafter, an optical path used when the imaging device 105 executes imaging is called an imaging optical path.

The imaging device 105 includes the imaging region 1051 in which the first optical image of the subject and the second optical image of the subject are formed in common. A plurality of pixels are disposed in a matrix shape in the imaging region 1051. The first optical image is formed by the light passing through the first optical path L1. The second optical image is formed by the light passing through the second optical path L2 different from the first optical path L1. The imaging device 105 acquires the first optical image and the second optical image by executing imaging.

The imaging device 105 executes imaging at a first imaging timing and acquires the first optical image formed by the light passing through the first optical system 101. The imaging device 105 executes imaging at a second imaging timing different from the first imaging timing and acquires the second optical image formed by the light passing through the second optical system 102. The imaging device 105 generates the first image on the basis of the first optical image formed in the imaging region 1051 and generates the second image on the basis of the second optical image formed in the imaging region 1051.

The imaging device 105 acquires the first optical image at one or more first imaging timings different from each other and generates one or more first images. In addition, the imaging device 105 acquires the second optical image at one or more second imaging timings different from each other and generates one or more second images. The imaging device 105 outputs the first image and the second image to a device control unit 107 included in the main body unit 2. The operation of the imaging device 105 is controlled by the device control unit 107. For example, a CCD image sensor or a CMOS image sensor can be used as the imaging device 105. The imaging device 105 is an example of the imaging device 16 shown in FIG. 1.

The optical path-switching unit 103 has a function of transmitting only light passing through one of the first optical path L1 and the second optical path L2 and has a function of blocking light passing through the other of the first optical path L1 and the second optical path L2. The optical path-switching unit 103 switches optical paths between the first optical path L1 and the second optical path L2, thus forming only any one of the first optical image and the second optical image in the imaging region 1051 of the imaging device 105.

The movement detection unit 106 has a function of detecting movement of an optical element. The optical element is a shielding unit 1031 included in the optical path-switching unit 103. In general, it can be said that the movement detection unit 106 detects movement of the distal end part 4. The movement detection unit 106 detects absolute movement of the optical element or detects relative movement of the optical element to the imaging device 16. The movement detection unit 106 may detect movement of the optical element by measuring the movement. The movement detection unit 106 is an example of the detection unit 12 shown in FIG. 1. Details of the movement detection unit 106 will be described later.

The main body unit 2 includes the device control unit 107, a frame memory 108, a measurement unit 109, a determination unit 110, and an optical-path control unit 111.

The device control unit 107 controls the entire endoscope device 1. The device control unit 107 executes optical path-switching control, first image-generation control, second image-generation control, and measurement control. The device control unit 107 notifies the optical-path control unit 111 of information of the imaging optical path used for imaging, thus executing the optical path-switching control. The optical-path control unit 111 controls the optical path-switching unit 103 on the basis of the information, thus controlling switching between the first optical path L1 and the second optical path L2. When the first optical path L1 is used, the device control unit 107 causes the imaging device 105 to acquire the first optical image and generate one or more first images on the basis of the first optical image by executing the first image-generation control. When the second optical path L2 is used, the device control unit 107 causes the imaging device 105 to acquire the second optical image and generate one or more second images on the basis of the second optical image by executing the second image-generation control. The device control unit 107 causes the measurement unit 109 to execute measurement by executing the measurement control. When the device control unit 107 receives a notification for executing the optical path-switching control again from the optical-path control unit 111, the device control unit 107 can repeat control of the measurement unit 109. The device control unit 107 is an example of the control unit 14 shown in FIG. 1.

The frame memory 108 stores the first image and the second image generated by the imaging device 105. The frame memory 108 is constituted as a volatile or nonvolatile memory. For example, the frame memory 108 is a volatile or nonvolatile memory such as a random-access memory (RAM), a dynamic random-access memory (DRAM), a static random-access memory (SRAM), an electrically erasable programmable read-only memory (EEPROM), or a flash memory. The endoscope device 1 may include a hard disk drive for recording the first image and the second image.

The measurement unit 109 measures at least one of the shape of a subject and the distance (subject distance) to the subject on the basis of the first image and the second image. For example, the shape of the subject is the distance between two arbitrary points on the subject, the area of the region constituted by three or more points on the subject, and the like. The subject distance is the distance from the distal end part 4 in which the imaging device 105 is disposed to the subject. The measurement unit 109 executes stereo measurement on the basis of triangulation using the parallax between two images.

The determination unit 110 has a function of determining whether or not the optical element moves by determining whether or not a shock has occurred in the distal end part 4 on the basis of the data output from the movement detection unit 106. For example, the determination unit 110 can determine whether or not a shock has occurred in the distal end part 4 by determining whether or not momentary movement exceeding a predetermined amount has occurred or a momentary force exceeding a predetermined amount has been added to the distal end part 4. The determination unit 110 is an example of the determination unit 13 shown in FIG. 1.

The optical-path control unit 111 has a function of controlling the optical path-switching unit 103. The optical-path control unit 111 is an example of the control unit 14 shown in FIG. 1.

The device control unit 107, the measurement unit 109, the determination unit 110, and the optical-path control unit 111 are constituted by at least one of a processor and a logic circuit. For example, a central processing unit (CPU), a digital signal processor (DSP), a graphics-processing unit (GPU), and the like can be used as a processor. For example, an application-specific integrated circuit (ASIC), a field-programmable gate array (FPGA), and the like can be used as a logic circuit. The device control unit 107, the measurement unit 109, the determination unit 110, and the optical-path control unit 111 may include one or a plurality of processors. The device control unit 107, the measurement unit 109, the determination unit 110, and the optical-path control unit 111 may include one or a plurality of logic circuits.

The computer of the endoscope device 1 may read a program and execute the read program. The program includes commands defining the operations of the device control unit 107, the measurement unit 109, the determination unit 110, and the optical-path control unit 111. In other words, the functions of the device control unit 107, the measurement unit 109, the determination unit 110, and the optical-path control unit 111 may be realized by software. The program, for example, may be provided by using a computer-readable storage medium such as a flash memory. In addition, the above-described program may be transmitted from a computer including a storage device or the like storing the program to the endoscope device 1 through a transmission medium or transmission waves in a transmission medium. The transmission medium transmitting the program is a medium having a function of transmitting information. The medium having the function of transmitting information includes a network (communication network) such as the Internet and a communication circuit line (communication line) such as a telephone line. In addition, the above-described program may also be a differential file (differential program) that can realize the above-described functions in combination with a program already recorded in the computer.

The operation unit 5 is a user interface that receives an instruction from a user. By operating the operation unit 5, a user inputs instructions necessary for controlling various operations of the entire endoscope device 1. The operation unit 5 outputs a signal indicating an instruction received from a user to the device control unit 107. For example, at least one of a button, a switch, a key, a mouse, a joystick, a touch pad, a track ball, and a touch panel is used as the operation unit 5.

The display unit 6 displays at least one of the first image and the second image. In addition, the display unit 6 displays operation control contents, measurement results, and the like. For example, the operation control contents are displayed as a menu. For example, at least one of a liquid crystal display and an organic electro-luminescence (EL) display is used as the display unit 6. In addition, the display unit 6 may be a touch panel display. In such a case, the operation unit 5 and the display unit 6 can be integrated.

The recording medium 7 stores the first image, the second image, the measurement results, and the like. For example, the recording medium 7 is a nonvolatile recording medium such as a flash memory. The recording medium 7 may be attachable to and detachable from the main body unit 2.

The power source 8 supplies power necessary for the endoscope device 1 to operate to each unit of the endoscope device 1. For example, a secondary battery (storage battery) such as a nickel-hydrogen battery or a lithium-ion battery can be used as the power source 8. In a case in which the endoscope device 1 is used outside, a secondary battery generally needs to be used. An external power source may be used. In FIG. 1, functions including a light source or the like used in a general endoscope are not shown.

The optical path-switching unit 103 is configured to transmit light passing through only one of the first optical path L1 and the second optical path L2 and is configured to block light passing through the other of the first optical path L1 and the second optical path L2. In this way, the optical path-switching unit 103 switches optical paths between the first optical path L1 and the second optical path L2 so that only any one of the first optical image and the second optical image is formed in the imaging region 1051 of the imaging device 105. For example, the optical path-switching unit 103 includes the shielding unit 1031 to be inserted into only any one of the first optical path L1 and the second optical path L2. The shielding unit 1031 is an example of the optical element 18 shown in FIG. 1.

When the optical path-switching unit 103 transmits light passing through the first optical path L1, the shielding unit 1031 is inserted into the second optical path L2 as shown in FIG. 5 and light passing through the second optical path L2 is blocked. At this time, the shielding unit 1031 is disposed at a second position on the second optical path L2. Accordingly, the first optical path L1 is used as the imaging optical path. On the other hand, when the optical path-switching unit 103 transmits light passing through the second optical path L2, the shielding unit 1031 is inserted into the first optical path L1 as shown in FIG. 6 and light passing through the first optical path L1 is blocked. At this time, the shielding unit 1031 is disposed at a first position on the first optical path L1. Accordingly, the second optical path L2 is used as the imaging optical path.

The optical path-switching unit 103 moves the shielding unit 1031 from the first position to the second position or from the second position to the first position, thus switching between the first optical path L1 and the second optical path L2. The optical path-switching unit 103 sets any one of the first optical path L1 and the second optical path L2 as the imaging optical path. The operation of the optical path-switching unit 103 is controlled by the optical-path control unit 111.

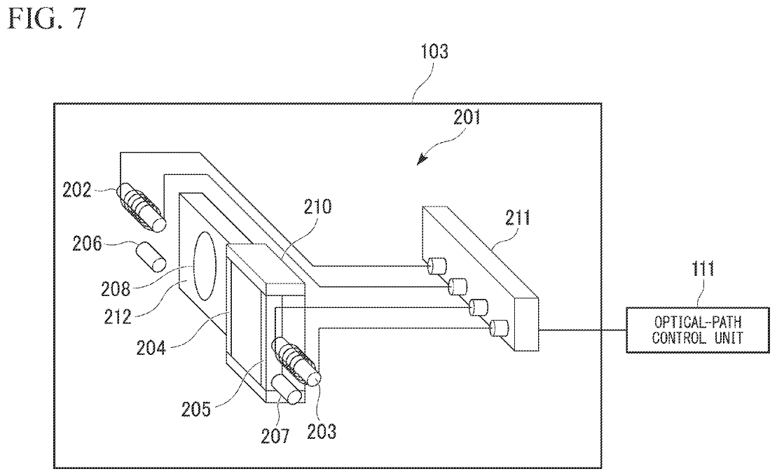

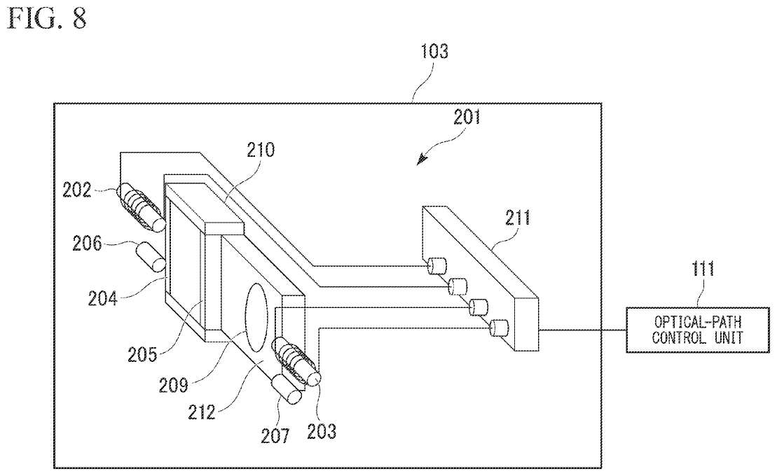

An example in which the optical path-switching unit 103 realizes an operation of switching between optical paths will be described. FIG. 7 and FIG. 8 show a configuration of a magnetic actuator 201 that is an example of the optical path-switching unit 103.

The magnetic actuator 201 shown in FIG. 7 and FIG. 8 includes a first electromagnet 202, a second electromagnet 203, a first permanent magnet 204, a second permanent magnet 205, a third permanent magnet 206, a fourth permanent magnet 207, a first opening 208, a second opening 209, a shielding unit 210, a current control unit 211, and a light selection unit 212. The shielding unit 210 corresponds to the shielding unit 1031 of the optical path-switching unit 103.

The first permanent magnet 204 and the second permanent magnet 205 are mounted on the shielding unit 210. For example, the first permanent magnet 204 and the second permanent magnet 205 are disposed so as to locate the central part of the shielding unit 210 between them. The first electromagnet 202 and the third permanent magnet 206 are disposed at positions facing the first permanent magnet 204. The second electromagnet 203 and the fourth permanent magnet 207 are disposed at positions facing the second permanent magnet 205.

The first electromagnet 202 is disposed so as to attract or repel the first permanent magnet 204 when a current flows in the first electromagnet 202. The second electromagnet 203 is disposed so as to attract or repel the second permanent magnet 205 when a current flows in the second electromagnet 203.

The first permanent magnet 204 turns its N-pole in the direction toward, for example, the first electromagnet 202. At this time, the third permanent magnet 206 turns its S-pole in the direction toward the first permanent magnet 204 so as to attract the first permanent magnet 204. The second permanent magnet 205 turns its S-pole in the direction toward, for example, the second electromagnet 203. At this time, the fourth permanent magnet 207 turns its N-pole in the direction toward the second permanent magnet 205 so as to attract the second permanent magnet 205.

The third permanent magnet 206 is able to generate a holding force to prevent the shielding unit 210 from moving when the moving direction of the shielding unit 210 matches the direction in which the gravitational acceleration acts. The moving direction of the shielding unit 210 indicates a direction in which the shielding unit 210 is able to move. In addition, the third permanent magnet 206 is able to generate a holding force to cause the shielding unit 210 to start moving when a first current flows in the first electromagnet 202. The first current causes the first electromagnet 202 to generate a magnetic field that repels the first permanent magnet 204.

The fourth permanent magnet 207 is able to generate a holding force to prevent the shielding unit 210 from moving when the moving direction of the shielding unit 210 matches the direction in which the gravitational acceleration acts. In addition, the fourth permanent magnet 207 is able to generate a holding force to cause the shielding unit 210 to start moving when a second current flows in the second electromagnet 203. The second current causes the second electromagnet 203 to generate a magnetic field that repels the second permanent magnet 205.

The following expression (1) shows a condition for preventing the shielding unit 210 from moving when the moving direction of the shielding unit 210 matches the direction in which the gravitational acceleration acts. It is possible to calculate the relationship of magnetic flux density between the first permanent magnet 204 and the third permanent magnet 206 by using the expression (1).

$${k}_{m} \frac{{B}_{2} {S}_{3} {B}_{3} {S}_{3}}{{d}^{2}} > mg \qquad ( 1 )$$

Here, B₂ indicates the magnetic flux density of the first permanent magnet 204, S₃ indicates the cross-sectional area of the third permanent magnet 206, B₃ indicates the magnetic flux density of the third permanent magnet 206, d indicates the distance between the first permanent magnet 204 and the third permanent magnet 206, kₘ indicates the Coulomb constant, m indicates the mass of the shielding unit 210, and g indicates the gravitational acceleration.

The following expression (2) shows a condition for causing the shielding unit 210 to start moving when the first current flows in the first electromagnet 202. The first current causes the first electromagnet 202 to generate a magnetic field that repels the first permanent magnet 204. It is possible to calculate the relationship of magnetic flux density between the first electromagnet 202, the first permanent magnet 204, and the third permanent magnet 206 by using the expression (2).

$${k}_{m} \frac{{B}_{2} {S}_{3} {B}_{3} {S}_{3}}{{d}^{2}} + mg < {k}_{m} \frac{{B}_{1} {S}_{1} {B}_{2} {S}_{1}}{{d}^{2}} \qquad ( 2 )$$

Here, B₁ indicates the magnetic flux density of the first electromagnet 202 and

S1 indicates the cross-sectional area of the first electromagnet 202. In addition, it is possible to calculate the magnetic flux density B₁ of the first electromagnet 202 by using the following expression (3).

B₁=μᵣμ₀nI (3)

Here, μᵣ indicates the permeability inside the first electromagnet 202, μ₀indicates the permeability in a vacuum, n indicates the number of turns of the coil of the first electromagnet 202, and I indicates the amount of current flowing in the first electromagnet 202.

It is possible to calculate the magnetic flux density necessary for each of the first permanent magnet 204 and the third permanent magnet 206 and calculate a design value of the first electromagnet 202 by using these expressions (1) to (3). Here, the expression (1) shows a minimum condition under which it is not assumed that the distal end part 4 moves. In fact, it is necessary that the value of the left side of the expression (1) not be close to the value of the right side of the expression (1). In fact, an optimal value needs to be adjusted by using the expressions (1) to (3), an available value of current in terms of design, and an available cross-sectional area of the magnet in terms of design.

It is also possible to define the relationship of magnetic flux density between the fourth permanent magnet 207 and the second electromagnet 203 for holding the shielding unit 210 as with the above.

The third permanent magnet 206 and the fourth permanent magnet 207 constitute a holding mechanism that holds the shielding unit 210 at the first position or the second position by using a predetermined amount of force. When the predetermined amount of force is added to the shielding unit 210, the shielding unit 210 moves.

The plate-like light selection unit 212 is disposed so as to overlap the shielding unit 210. The light selection unit 212 selects one of the light passing through the first optical system 101 and the light passing through the second optical system 102 and projects the selected light on the imaging region 1051 of the imaging device 105.

The first opening 208 and the second opening 209 are formed in the light selection unit 212. The first opening 208 and the second opening 209 are formed along the moving direction of the shielding unit 210. The first opening 208 overlaps the first optical path L1 shown in FIG. 1. The first opening 208 is able to transmit light when the shielding unit 210 moves to the second electromagnet 203 side. The second opening 209 overlaps the second optical path L2 shown in FIG. 1. The second opening 209 is able to transmit light when the shielding unit 210 moves to the first electromagnet 202 side.

When the shielding unit 210 is moved by the first electromagnet 202 and the second electromagnet 203, the shielding unit 210 moves to a position so as to shield one of the first opening 208 and the second opening 209.

FIG. 7 shows a state in which the shielding unit 210 shields the second opening 209. The shielding unit 210 is disposed at the second position so as to shield the second opening 209. The shielding unit 210 does not shield the first opening 208. The light passing through the first optical system 101 passes through the first opening 208. The light is incident on the imaging region 1051 of the imaging device 105. The light passing through the second optical system 102 is blocked by the shielding unit 210. The light is not incident on the imaging region 1051 of the imaging device 105. Therefore, the light selection unit 212 selects the light passing through the first optical system 101.

FIG. 8 shows a state in which the shielding unit 210 shields the first opening 208. The shielding unit 210 is disposed at the first position so as to shield the first opening 208. The shielding unit 210 does not shield the second opening 209. The light passing through the second optical system 102 passes through the second opening 209. The light is incident on the imaging region 1051 of the imaging device 105. The light passing through the first optical system 101 is blocked by the shielding unit 210. The light is not incident on the imaging region 1051 of the imaging device 105. Therefore, the light selection unit 212 selects the light passing through the second optical system 102.

The current control unit 211 controls a current flowing in the first electromagnet 202 and the second electromagnet 203. The current control unit 211 causes a current to flow in the first electromagnet 202 and the second electromagnet 203 and causes the first electromagnet 202 and the second electromagnet 203 to generate a magnetic field, thus realizing a first state or a second state. In the first state, the first electromagnet 202 and the first permanent magnet 204 repel each other and the second electromagnet 203 and the second permanent magnet 205 attract each other. In the first state, the shielding unit 210 is disposed at the second position so as to shield the second opening 209. In the second state, the first electromagnet 202 and the first permanent magnet 204 attract each other and the second electromagnet 203 and the second permanent magnet 205 repel each other. In the second state, the shielding unit 210 is disposed at the first position so as to shield the first opening 208. The current control unit 211 is able to move the shielding unit 210 by controlling these two states.

The current control unit 211 stops supplying a current to the first electromagnet 202 and the second electromagnet 203, thus realizing a third state. In the third state, the shielding unit 210 is held by the third permanent magnet 206 or the fourth permanent magnet 207, and the shielding unit 210 stands still. In this way, the current control unit 211 can suppress power consumption and can suppress heat generation caused by a current.

The current control unit 211 receives a signal for switching between optical paths from the optical-path control unit 111 and controls a current flowing in the first electromagnet 202 and the second electromagnet 203. In this way, the current control unit 211 can switch between positions of the shielding unit 210. The magnetic actuator 201 moves the shielding unit 210 from the first position to the second position or from the second position to the first position.

Details of the movement detection unit 106 will be described. The movement detection unit 106 detects movement of the optical element. In the second embodiment, the movement detection unit 106 detects absolute movement of the optical element.

In the second embodiment, an acceleration sensor of one axis is used as the movement detection unit 106. Other than this, for example, an acceleration sensor or a gyro sensor of two or three axes can be used as the movement detection unit 106, and an inertial measurement unit (IMU) or the like including both the acceleration sensor and the gyro sensor can be used as the movement detection unit 106. In the second embodiment, the movement detection unit 106 detects the moving direction of the shielding unit 210 of the optical path-switching unit 103.

The movement detection unit 106 generates an analog value indicating the amount of the detected movement. The movement detection unit 106 converts the analog value into a digital value by using an A/D converter and outputs a digital signal indicating the digital value to the determination unit 110. As a method of transmission between the movement detection unit 106 and the determination unit 110, wired communication using a communication line such as an electric wire or an optical fiber cable is used, or wireless communication such as a wireless LAN or a wireless MAN is used. The movement detection unit 106 does not need to be disposed near the optical path-switching unit 103.

Details of the determination unit 110 will be described. In the following example, the determination unit 110 determines the amount of force added to the optical element, thus determining the amount of movement of the optical element. Specifically, the determination unit 110 determines whether or not the force exceeding a predetermined amount is added to the optical element, thus determining whether or not the optical element moves from the first position or the second position. For example, the predetermined amount is the amount of force necessary for moving the optical element from the first position to the second position or moving the optical element from the second position to the first position. As an example in which the determination unit 110 determines that the optical element moves from the first position or the second position, an example in which the determination unit 110 determines whether or not a shock has occurred will be described.

The digital signal output from the movement detection unit 106 is input into the determination unit 110. Since the digital signal includes components of the gravity acting in the direction of an acceleration sensor of one axis, the determination unit 110 extracts components of the gravitational acceleration from the digital signal by using a low-pass filter. The determination unit 110 subtracts a signal indicating the extracted gravitational acceleration from the digital signal. In this way, the determination unit 110 can reduce the influence of the gravitational acceleration in the determination of shock.

In addition, the determination unit 110 uses a low-pass filter and processes the digital signal from which the signal indicating the gravitational acceleration has been subtracted in order to reduce the influence of noise. At this time, the low-pass filter for reducing noise is designed to transmit a signal having a higher frequency compared to the low-pass filter for extracting the gravitational acceleration. In this way, the determination unit 110 can acquire the gravitational acceleration and the acceleration in the moving direction of the shielding unit 210 of the optical path-switching unit 103 from the digital signal obtained from the movement detection unit 106.

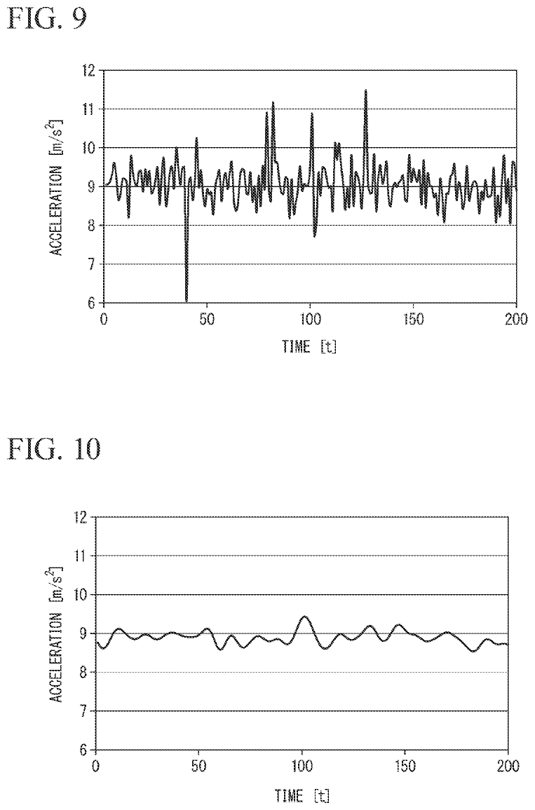

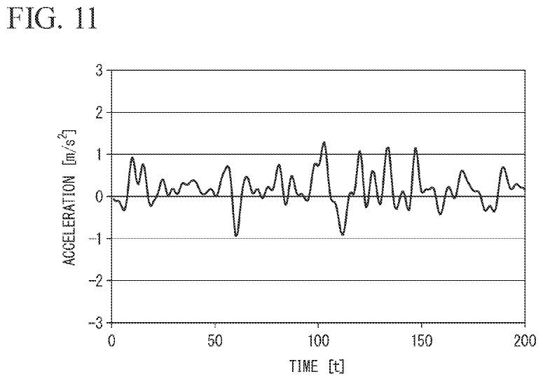

FIG. 9 shows a waveform of the digital signal input into the determination unit 110. FIG. 10 shows gravitational components (gravitational acceleration) indicated by the components extracted from the digital signal. FIG. 11 shows the acceleration indicated by the components obtained by eliminating components of the gravitational acceleration and noise from the digital signal. In each of FIG. 9, FIG. 10, and FIG. 11, the horizontal axis indicates time and the vertical axis indicates an acceleration.

The determination unit 110 determines whether or not a shock has occurred in the distal end part 4 on the basis of the data of the gravitational acceleration and the acceleration acquired by using the digital filter. For example, the determination unit 110 uses the following determination method.

The position of the shielding unit 210 is fixed by the holding force generated by the first permanent magnet 204 and the third permanent magnet 206 attracting each other or is fixed by the holding force generated by the second permanent magnet 205 and the fourth permanent magnet 207 attracting each other. This holding force F_{r is} calculated by using the following expression (4).

$${F}_{r} = {k}_{m} \frac{{B}_{2} {S}_{3} {B}_{3} {S}_{3}}{{d}^{2}} \qquad ( 4 )$$

When a force exceeding the holding force Fᵣ shown in the expression (4) occurs, the shielding unit 210 unintentionally moves. The gravity F_{g} shown in the following expression (5) and the inertial force Fᵢ shown in the following expression (6) act on the shielding unit 210. The gravity F_{g} shown in the expression (5) indicates the gravity acting in the moving direction of the shielding unit 210. Here, g in the expression (5) indicates the gravitational acceleration indicated by the components extracted from the digital signal. The inertial force F, shown in the expression (6) indicates a force generated on the basis of the acceleration when the distal end part 4 suddenly moves. Here, a in the expression (6) indicates the acceleration indicated by the components obtained by eliminating the gravitational acceleration and the noise from the digital signal.

F_{g=mg} (5)

Fᵢ=ma (6)

Thus, the shielding unit 210 moves when the condition shown in the following expression (7) is met. In other words, the shielding unit 210 moves when the sum of the gravity and the inertial force acting on the shielding unit 210 exceeds the holding force Fᵣ shown in the expression (4).

$${k}_{m} \frac{{B}_{2} {S}_{3} {B}_{3} {S}_{3}}{{d}^{2}} < mg + ma \qquad ( 7 )$$

The determination unit 110 calculates the holding force Fᵣ shown in the expression (4). The determination unit 110 determines whether or not a shock has occurred on the basis of the data of the gravitational acceleration and the acceleration acting in the moving direction of the shielding unit 210 by using the expression (7). At this time, the holding force Fᵣ shown in the expression (4) is used as a threshold value. If the condition shown in the expression (7) is met, the determination unit 110 determines that a shock has occurred. If the condition shown in the expression (7) is not met, the determination unit 110 determines that a shock has not occurred. When the determination unit 110 determines that a shock has occurred, the determination unit 110 outputs a shock occurrence signal to the optical-path control unit 111.

Even when an acceleration sensor of two or three axes is used, a determination method is similar to that described above. In addition, the determination unit 110 can execute the determination more securely by using an acceleration in a direction other than the moving direction of the shielding unit 210. In a case in which a gyro sensor is used, the determination unit 110 calculates an acceleration by calculating the difference in angular velocity. In addition, the determination unit 110 can determine a shock by detecting movement that generates an inertial force in the moving direction of the shielding unit 210.

In the above-described example, the determination unit 110 determines that a momentary force exceeding the predetermined amount has occurred. When momentary movement exceeding a predetermined amount has occurred, a momentary force exceeding the predetermined amount occurs. Therefore, it can be said that the determination unit 110 determines that the momentary movement exceeding the predetermined amount has occurred.

Details of the optical-path control unit 111 will be described. The optical-path control unit 111 receives information corresponding to the operation mode used for imaging from the device control unit 107. The optical-path control unit 111 controls the optical path-switching unit 103 on the basis of the information, thus disposing the shielding unit 210 at any one of the first position and the second position. In this way, the optical-path control unit 111 switches between the first optical path L1 and the second optical path L2.

For example, an observation mode and a measurement mode are defined as the operation mode. In the observation mode, only the first optical path L1 or the second optical path L2, which is set in advance, is used as the imaging optical path. In the measurement mode, the imaging device 105 acquires a first optical image formed by light passing through the first optical path L1 at one or more first imaging timings and generates one or more first images. In addition, in the measurement mode, the imaging device 105 acquires a second optical image formed by light passing through the second optical path L2 at one or more second imaging timings and generates one or more second images.

In addition, the optical-path control unit 111 receives the shock occurrence signal output from the determination unit 110 and controls the optical path-switching unit 103. For example, when the optical-path control unit 111 receives the shock occurrence signal while the observation mode is set in the endoscope device 1, the optical-path control unit 111 controls the optical path-switching unit 103 so that the first optical path L1 or the second optical path L2, which is set in advance, is used as the imaging optical path. For example, when the optical-path control unit 111 receives the shock occurrence signal while the measurement mode is set in the endoscope device 1, the optical-path control unit 111 repeats the operation performed in the measurement mode from the beginning. After the optical-path control unit 111 receives the shock occurrence signal from the determination unit 110, the optical-path control unit 111 executes the above-described control. In this way, the optical-path control unit 111 can deal with an abnormal operation by the optical path-switching unit 103 caused by a shock and can reset the state of the optical path-switching unit 103 to be a state of executing a suitable operation for the observation mode or the measurement mode.

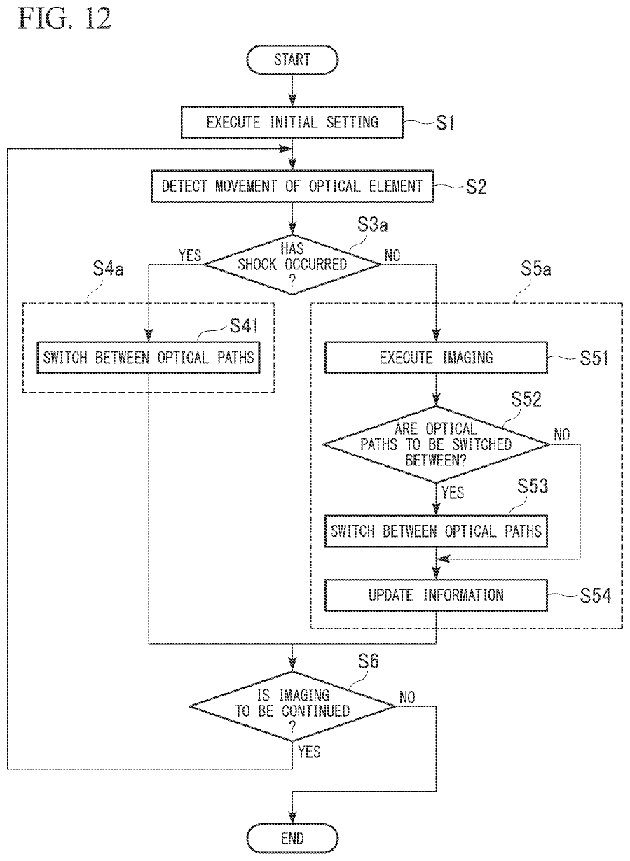

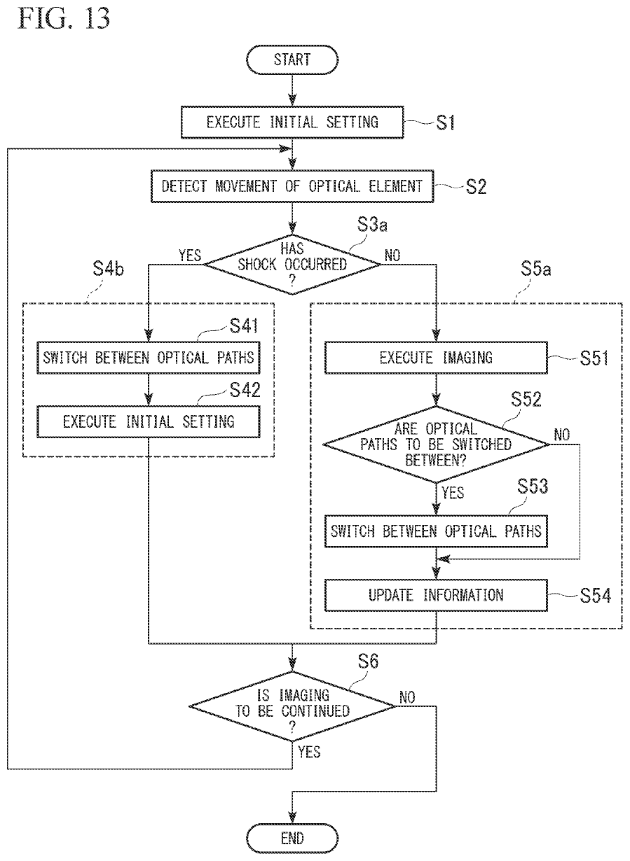

An operation of the endoscope device 1 in the observation mode or the measurement mode will be described with reference to FIG. 12 and FIG. 13. Two or more images are generated in the image-generation sequence of the observation mode. Two or more images are generated in the image-generation sequence of the measurement mode.

FIG. 12 shows a procedure of processing executed by the endoscope device 1 for switching between optical paths and continuing the image-generation sequence when a shock occurs. In the processing shown in FIG. 12, imaging is resumed in order to execute the rest of the image-generation sequence after a shock occurs.

The device control unit 107 executes initial setting. At this time, the device control unit 107 selects sequence information indicating details of the image-generation sequence for generating images. For example, the sequence information indicates the number of frames in which the imaging device 105 executes imaging and indicates a position of the optical element 18 in each frame. One or more pieces of the sequence information are stored on the memory of the endoscope device 1 in advance. For example, when a user designates the observation mode or the measurement mode, the device control unit 107 selects the sequence information corresponding to the mode selected by the user and outputs the sequence information to the optical-path control unit 111. In addition, the device control unit 107 initializes an imaging frame number indicating the number of frames in which imaging is executed. For example, the initialized imaging frame number indicates zero. The optical-path control unit 111 executes the disposition control and moves the shielding unit 210 of the optical path-switching unit 103 to the position indicated by the sequence information (Step S1).

After Step S1, the movement detection unit 106 detects movement of the optical element and outputs a digital signal indicating the detected movement to the determination unit 110 (Step S2).

After Step S2, the determination unit 110 determines whether or not a shock has occurred on the basis of the digital signal output from the movement detection unit 106 (Step S3a).

When the determination unit 110 determines that a shock has occurred in Step S3a, the determination unit 110 outputs the shock occurrence signal to the optical-path control unit 111. The optical-path control unit 111 executes the restoration control (Step S4a). In the restoration control, the following Step S41 is executed.

The optical-path control unit 111 controls the current control unit 211 of the optical path-switching unit 103 on the basis of the sequence information and the imaging frame number. The current control unit 211 moves the shielding unit 210. In this way, the optical-path control unit 111 moves the shielding unit 210 to the position indicated by the sequence information and switches between optical paths (Step S41). At this time, the imaging optical path is changed to a correct path in the image-generation sequence.

For example, when the correct path is the first optical path L1, the shielding unit 210 is disposed at the second position so as to shield the second opening 209. When a shock occurs, there is a possibility that the shielding unit 210 moves to the first position so as to shield the first opening 208. When the shielding unit 210 is disposed at the first position immediately before Step S41 is executed, the optical-path control unit 111 moves the shielding unit 210 to the second position in Step S41. When the shielding unit 210 is disposed at the second position immediately before Step S41 is executed, the optical-path control unit 111 does not need to move the shielding unit 210. Since the endoscope device 1 according to the second embodiment does not include a mechanism for confirming the position of the shielding unit 210, the optical-path control unit 111 executes control of moving the shielding unit 210 to the second position in the above-described example.

When the determination unit 110 determines that a shock has not occurred in Step S3a, the determination unit 110 outputs a determination result to the device control unit 107. The device control unit 107 executes the normal control (Step S5a). In the normal control, the following Steps S51 to S54 are executed.

The device control unit 107 causes the imaging device 105 to execute imaging The imaging device 105 acquires the first optical image or the second optical image and generates the first image or the second image (Step S51).

After Step S51, the device control unit 107 determines whether or not to switch between optical paths on the basis of the sequence information and the imaging frame number (Step S52). For example, when the sequence information indicates that the imaging optical path in the next frame is the same as the imaging optical path in the previous frame, the device control unit 107 determines not to switch between optical paths. This corresponds to the observation mode. When the sequence information indicates that the imaging optical path in the next frame is different from the imaging optical path in the previous frame, the device control unit 107 determines to switch between optical paths. This corresponds to the measurement mode.

When the device control unit 107 determines not to switch between optical paths in Step S52, Step S54 is executed. In this case, the optical-path control unit 111 does not switch between optical paths.

When the device control unit 107 determines to switch between optical paths in Step S52, the device control unit 107 instructs the optical-path control unit 111 to switch between optical paths. The optical-path control unit 111 switches between optical paths by executing similar processing to Step S41 (Step S53).

After Step S53, the device control unit 107 updates the imaging frame number by increasing the imaging frame number by one (Step S54).

After Step S41 or Step S54, the device control unit 107 determines whether or not to continue imaging on the basis of the sequence information and the imaging frame number (Step S6). For example, when the imaging frame number is less than a predetermined number of times indicated by the sequence information, the imaging device 105 has not executed imaging the predetermined number of times. In this case, the device control unit 107 determines to continue imaging. When the imaging frame number is the same as the predetermined number of times, the imaging device 105 has executed imaging the predetermined number of times. In this case, the device control unit 107 determines not to continue imaging

When the device control unit 107 determines to continue imaging in Step S6, Step S2 is executed. When the device control unit 107 determines not to continue imaging in Step S6, the processing shown in FIG. 12 is completed.

In a case in which the processing shown in FIG. 12 is executed only in the observation mode, Step S52 and Step S53 do not need to be executed. In other words, after Step S51, Step S54 may be executed without executing Step S52 and Step S53.

There is a possibility that a shock occurs and the restoration control is executed before the imaging device 105 generates an image in accordance with the image-generation sequence. Alternatively, there is a possibility that a shock occurs and the restoration control is executed after the imaging device 105 generates some of two or more images scheduled to be generated in the image-generation sequence. The optical-path control unit 111 executes Step S41 in the restoration control.

After Step S41, the device control unit 107 causes the imaging device 105 to execute imaging in Step S51 in a state in which the shielding unit 210 is disposed at a correct position. In this way, the device control unit 107 causes the imaging device 105 to resume imaging in order to generate the rest of the two or more images scheduled to be generated in the image-generation sequence. Even when the determination unit 110 determines that a shock has occurred in Step S3a, the imaging device 105 continues imaging in accordance with the image-generation sequence. When the operation mode is the measurement mode, the imaging device 105 continues stereo imaging.

In the processing shown in FIG. 12, after the imaging device 105 starts stereo imaging, the optical-path control unit 111 executes the restoration control (Step S4a) in a predetermined state. The predetermined state is a state in which the determination unit 110 determines that the shielding unit 210 moves from the first position or the second position after the disposition control is executed and before the disposition control is executed next. In Step S41, the optical-path control unit 111 disposes the shielding unit 210 at the position at which the shielding unit 210 was disposed through the disposition control executed immediately before the predetermined state occurs. Alternatively, the optical-path control unit 111 disposes the shielding unit 210 at the position at which the shielding unit 210 is scheduled to be disposed through the disposition control to be executed next.

Positions at which the shielding unit 210 is disposed and the number of times the imaging device 105 generates each of the first image and the second image are set in advance as the sequence information. When the determination unit 110 determines that a shock has occurred, the optical-path control unit 111 moves the shielding unit 210 to a predetermined position necessary for the imaging device 105 to generate the first image or the second image. The predetermined position is any one of the first position and the second position. After the shielding unit 210 moves to the predetermined position, the device control unit 107 causes the imaging device 105 to continue imaging. When the operation mode is the measurement mode, the device control unit 107 causes the imaging device 105 to continue stereo imaging.

FIG. 13 shows a procedure of processing executed by the endoscope device 1 for switching between optical paths and repeating the image-generation sequence when a shock occurs. In the processing shown in FIG. 13, imaging is resumed from the beginning of the image-generation sequence when a shock occurs. The same processing as that shown in FIG. 12 will not be described.

When the determination unit 110 determines that a shock has occurred in Step S3a, the determination unit 110 outputs the shock occurrence signal to the optical-path control unit 111. The optical-path control unit 111 executes the restoration control (Step S4b). In the restoration control, the following Step S41 and Step S42 are executed.

The optical-path control unit 111 moves the shielding unit 210 to the same position (initial position) as that at which the shielding unit 210 is disposed in Step S1 in order to repeat imaging from the beginning of the image-generation sequence. In other words, the optical-path control unit 111 moves the shielding unit 210 to the initial position at which the shielding unit 210 is disposed at the beginning of the image-generation sequence. Specifically, the optical-path control unit 111 controls the current control unit 211 on the basis of the sequence information. The current control unit 211 moves the shielding unit 210. In this way, the optical-path control unit 111 moves the shielding unit 210 to the position indicated by the sequence information and switches between optical paths (Step S41).

For example, the optical-path control unit 111 moves the shielding unit 210 to the second position so as to shield the second opening 209 in Step S1 in order to use the first optical path L1 as the imaging optical path. Thereafter, the optical-path control unit 111 moves the shielding unit 210 to the first position so as to shield the first opening 208 in Step S53 in order to use the second optical path L2 as the imaging optical path. The endoscope device 1 according to the second embodiment does not include a mechanism for confirming the position of the shielding unit 210. When the determination unit 110 determines that a shock has occurred in Step S3a, the optical-path control unit 111 executes control of moving the shielding unit 210 to the second position in Step S41 regardless of the actual position of the shielding unit 210.

After Step S41, the device control unit 107 executes the initial setting. At this time, the device control unit 107 selects the same sequence information as that selected in Step S1 and outputs the sequence information to the optical-path control unit 111. In addition, the device control unit 107 initializes the imaging frame number indicating the number of frames in which imaging is executed. For example, the initialized imaging frame number indicates zero (Step S42). After Step S42 is executed, Step S6 is executed.

In a case in which the processing shown in FIG. 13 is executed only in the measurement mode, Step S52 does not need to be executed. In other words, after Step S51, Step S53 may be executed without executing Step S52.

There is a possibility that a shock occurs and the restoration control is executed before the imaging device 105 generates an image in accordance with the image-generation sequence. Alternatively, there is a possibility that a shock occurs and the restoration control is executed after the imaging device 105 generates some of two or more images scheduled to be generated in the image-generation sequence. The optical-path control unit 111 executes Step S41 and Step S42 in the restoration control.

After Step S42, the device control unit 107 causes the imaging device 105 to execute imaging in Step S51 in a state in which the shielding unit 210 is disposed at the initial position. In this way, the device control unit 107 causes the imaging device 105 to start stereo imaging in order to generate all the two or more images scheduled to be generated in the image-generation sequence. The imaging device 105 stops stereo imaging, which is started before the determination in Step S3a is executed, and resumes the stereo imaging from the beginning.

In the processing shown in FIG. 13, the optical-path control unit 111 disposes the shielding unit 210 at the initial position, which is set in advance, among the first position and the second position by executing the disposition control in Step S1. The device control unit 107 causes the imaging device 105 to start stereo imaging in Step S51. After the imaging device 105 starts the stereo imaging, the optical-path control unit 111 executes the restoration control (Step S4b) in a predetermined state. The predetermined state is a state in which the determination unit 110 determines that the shielding unit 210 moves from the first position or the second position after the disposition control is executed and before the disposition control is executed next. The optical-path control unit 111 executes the disposition control of disposing the shielding unit 210 at the initial position in Step S41. The device control unit 107 causes the imaging device 105 to start the stereo imaging in Step S51.

The optical-path control unit 111 may execute the disposition control of disposing the shielding unit 210 at any one of the first position and the second position in Step S41. The position at which the shielding unit 210 is to be disposed is the same as or different from the initial position. After the shielding unit 210 is disposed at the first position or the second position, the device control unit 107 may cause the imaging device 105 to start the stereo imaging in Step S51.

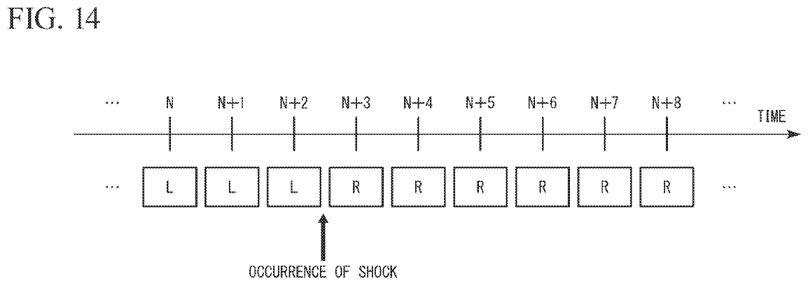

FIGS. 14 to 18 show specific examples of the image-generation sequence. In FIGS. 14 to 18, the horizontal axis indicates time. In FIGS. 14 to 18, the first image is shown as “L” and the second image is shown as “R.” In FIGS. 14 to 18, frame numbers are shown on the upper side of the axis indicating passage of time. The character N included in the frame number indicates an integer of one or more.

FIG. 14 shows an example of the image-generation sequence in an endoscope device that does not include the determination unit 110 and does not execute similar control to that executed by the optical-path control unit 111. In the example shown in FIG. 14, the operation mode is the observation mode.

An imaging device generates the first image “L” from the N-th frame to the (N+2)-th frame. A shock occurs after transfer of image data is completed in the (N+2)-th frame. Therefore, the imaging device generates the second image “R” in the (N+3)-th frame and each of the following frames. As described above, in the endoscope device not including the determination unit 110, generation of unintentional images continues in a case in which a different image from the scheduled image is generated. Therefore, different images from those intended by a user continue to be displayed.

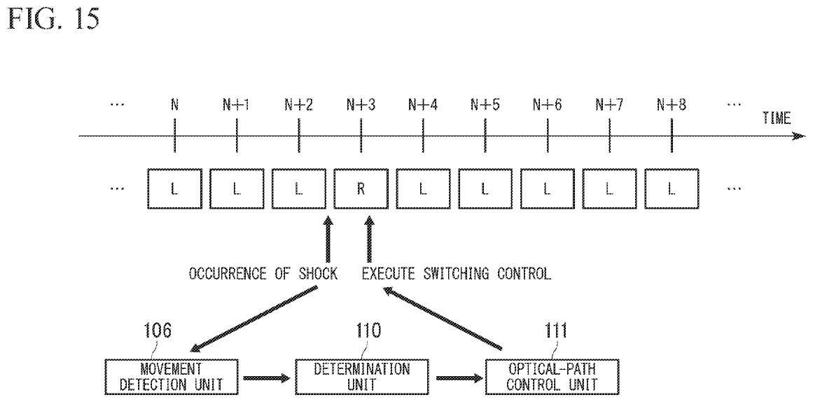

FIG. 15 shows an example of the image-generation sequence in a case in which the determination unit 110 executes determination related to shock and the optical-path control unit 111 executes switching control. In the example shown in FIG. 15, the operation mode is the observation mode.

Imaging is started as with the example shown in FIG. 14. The movement detection unit 106 detects movement of the optical element in each frame. A shock occurs at the same timing as that shown in FIG. 14. In the example shown in FIG. 15, the determination unit 110 detects a shock and the optical-path control unit 111 executes switching control after transfer of image data is completed in the (N+2)-th frame. The optical-path control unit 111 switches between optical paths after transfer of image data is completed in the (N+3)-th frame. The imaging device 105 generates the second image “R” in the (N+3)-th frame and generates the first image “L” in the (N+4)-th frame and each of the following frames.

Since the optical-path control unit 111 switches between optical paths, the imaging device 105 can generate appropriate images in the (N+4)-th frame and each of the following frames. Even when a different image from the scheduled image is generated, the endoscope device 1 can change the imaging optical path to a predetermined optical path necessary for observation and can continue to generate appropriate images.

When the determination unit 110 determines that a shock has occurred, the device control unit 107 may execute control of preventing an unintentional image from being displayed. For example, when the determination unit 110 determines that a shock has occurred, the device control unit 107 prevents the display unit 6 from displaying an image generated in the frame during which the shock occurs. The device control unit 107 causes the display unit 6 to display an image generated in each of the frames following the frame during which the shock occurs. The device control unit 107 may cause the display unit 6 to display an image generated in the frame that is one frame before the frame during which the shock occurs instead of an image generated in the frame during which the shock occurs.

In the example shown in FIG. 15, the device control unit 107 prevents the display unit 6 from displaying an image generated in the (N+3)-th frame. The device control unit 107 causes the display unit 6 to display an image generated in the (N+4)-th frame and each of the following frames. The display unit 6 continues to display only the first image “L.” Therefore, a user can continue observation without knowing that a different image from the scheduled image is generated.

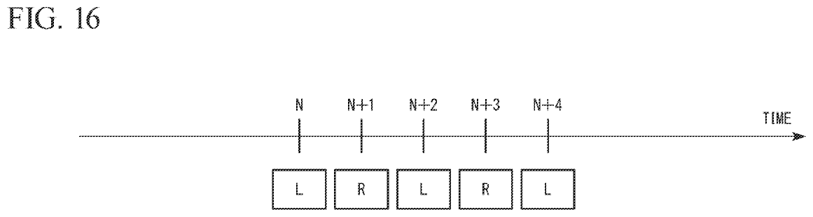

FIG. 16 shows an example of the image-generation sequence for generating an image necessary for the measurement unit 109. In the example shown in FIG. 16, the operation mode is the measurement mode.

In the example shown in FIG. 16, the imaging device 105 generates the first image “L” in each of the N-th frame, the (N+2)-th frame, and the (N+4)-th frame and generates the second image “R” in each of the (N+1)-th frame and the (N+3)-th frame.

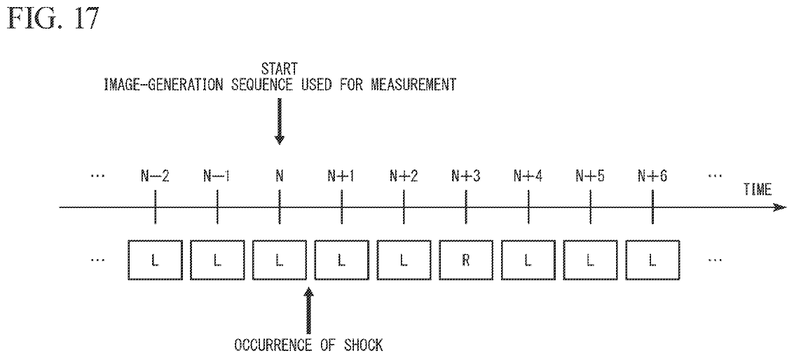

FIG. 17 shows an example of the image-generation sequence in an endoscope device that does not include the determination unit 110 and does not execute similar control to that executed by the optical-path control unit 111. In the example shown in FIG. 17, the operation mode is first set to a predetermined mode (for example, the observation mode) and then is changed to the measurement mode.

An imaging device generates the first image “L” in each of the (N−2)-th frame and the (N−1)-th frame. Thereafter, the operation mode is changed to the measurement mode, and the image-generation sequence used for measurement is started in the N-th frame. The imaging device generates the first image “L” in the N-th frame. A shock occurs after transfer of image data is completed in the N-th frame. If a shock does not occur, the imaging device generates the second image “R” in the (N+1)-th frame. Since a shock has occurred, the imaging device generates the first image “L” in the (N+1)-th frame. The imaging device generates the first image “L” or the second image “R” in accordance with the image-generation sequence in each of the (N+2)-th to the (N+4)-th frames.

The device control unit 107 outputs an image generated in each of the N-th to the (N+4)-th frames from the frame memory 108 to the measurement unit 109. In the example shown in FIG. 17, the image group input into the measurement unit 109 is different from that in the image-generation sequence shown in FIG. 16. Therefore, the measurement unit 109 is unable to correctly execute measurement.

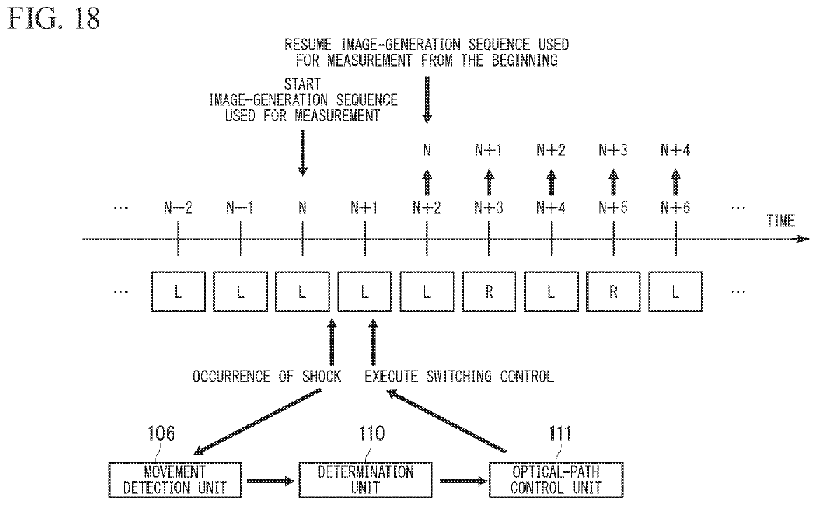

FIG. 18 shows an example of the image-generation sequence in a case in which the determination unit 110 executes determination related to shock and the optical-path control unit 111 executes switching control. In the example shown in FIG. 18, the operation mode is first set to a predetermined mode (for example, the observation mode) and then is changed to the measurement mode.

As with the example shown in FIG. 17, the imaging device 105 generates the first image “L” in each of the (N−2)-th frame and the (N−1)-th frame, and the image-generation sequence used for measurement is started in the N-th frame. The imaging device 105 generates the first image “L” in the N-th frame. The movement detection unit 106 detects movement of the optical element in each frame. A shock occurs at the same timing as that shown in FIG. 17. In the example shown in FIG. 18, the determination unit 110 detects a shock and the optical-path control unit 111 executes switching control after transfer of image data is completed in the N-th frame. The optical-path control unit 111 sets the imaging optical path in the (N+2)-th frame to the same imaging optical path as that in the N-th frame. The image-generation sequence is repeated from the (N+2)-th frame to the (N+6)-th frame. The imaging device 105 generates the first image “L” or the second image “R” in accordance with the image-generation sequence in each of the (N+2)-th to the (N+6)-th frames.

The optical-path control unit 111 sets the imaging optical path to the same imaging optical path as that first set in the image-generation sequence, and the image-generation sequence is resumed from the beginning. Therefore, the endoscope device 1 can generate images necessary in the measurement unit 109. The measurement unit 109 can correctly execute measurement.

In the second embodiment, the optical-path control unit 111 controls the position of the shielding unit 210 on the basis of the result of determination related to shock. Therefore, the endoscope device 1 can suppress the occurrence of a situation in which the imaging device 105 generates an image with the shielding unit 210 being disposed at an incorrect position.

When the determination unit 110 determines that a shock has occurred in the observation mode, the optical-path control unit 111 disposes the shielding unit 210 to the position at which the shielding unit 210 was disposed through the disposition control executed last. In other words, the optical-path control unit 111 disposes the shielding unit 210 to the position at which the shielding unit 210 should be disposed at present. Alternatively, the optical-path control unit 111 disposes the shielding unit 210 to the position at which the shielding unit 210 is scheduled to be disposed through the disposition control to be executed next. When the shielding unit 210 is disposed at an incorrect position due to a shock, the optical-path control unit 111 can move the shielding unit 210 to a correct position. After the shielding unit 210 moves to a correct position, the endoscope device 1 can resume generation of images for observation.

When the determination unit 110 determines that a shock has occurred in the measurement mode, the optical-path control unit 111 disposes the shielding unit 210 to the initial position and the device control unit 107 causes the imaging device 105 to start stereo imaging The endoscope device 1 can resume generation of images for measurement from the beginning.

### Third Embodiment

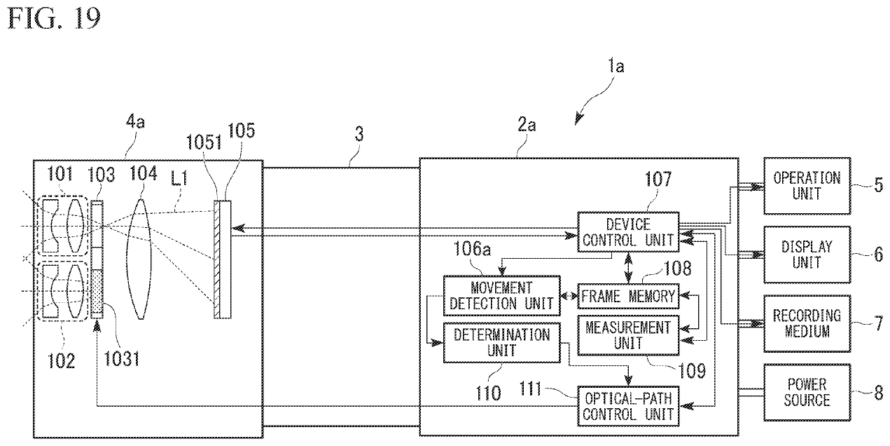

FIG. 19 shows a configuration of an endoscope device 1a according to a third embodiment of the present invention. The same configuration as that shown in FIG. 5 and FIG. 6 will not be described.

The main body unit 2 shown in FIG. 5 and FIG. 6 is changed to a main body unit 2a. The distal end part 4 shown in FIG. 5 and FIG. 6 is changed to a distal end part 4a. The distal end part 4a does not include the movement detection unit 106, and the main body unit 2a includes a movement detection unit 106a.

The device control unit 107 outputs a storage position (address) of each of one or more first images and one or more second images in the frame memory 108 to the movement detection unit 106a. The movement detection unit 106a reads at least one of the first image and the second image from the frame memory 108 on the basis of the storage position output from the device control unit 107. The movement detection unit 106a detects movement of the optical element on the basis of at least one of the first image and the second image.

The first optical system 101 and the second optical system 102 are apart from each other. Therefore, the parallax occurs between the first optical image formed by light passing through the first optical system 101 and the second optical image formed by light passing through the second optical system 102. When the shielding unit 210 of the optical path-switching unit 103 unintentionally moves, movement of a subject image occurs in an image on the basis of the parallax between the first optical image and the second optical image. The direction of the movement occurring on the basis of the parallax is the same as the horizontal direction of the image.

In the third embodiment, the movement detection unit 106a detects relative movement of the optical element to the imaging device 105. For example, the movement detection unit 106a uses two consecutive images and detects movement of a subject image. In this way, the movement detection unit 106a detects movement of the optical element.

The movement detection unit 106a is able to use an image-matching technique in order to detect movement of the subject image. The movement detection unit 106a is able to use, for example, template matching, feature-point matching, phase-only correlation, and the like as the image-matching technique. The movement detection unit 106a is able to use an index of similarity such as the sum of absolute difference (SAD), the sum of squared difference (SSD), the normalized cross-correlation (NCC), or the zero-means normalized cross-correlation (ZNCC) in the template matching. In addition, the movement detection unit 106a is able to use a feature quantity such as the binary robust invariant scalable keypoints (BRISK), the oriented features from accelerated segment test (FAST) and rotated binary robust independent elementary features (BRIEF) (ORB), the KAZE, or the accelerated KAZE (AKAZE) in the feature-point matching. The movement detection unit 106a detects the amount (movement amount) of movement on an image as movement of the subject image.

The movement detection unit 106a outputs the result of detecting the movement amount on an image to the determination unit 110. The movement detection unit 106a may convert the movement amount on an image into an estimation value of movement of the optical element and may output the estimation value to the determination unit 110. Alternatively, the movement detection unit 106a may convert the movement amount output from the determination unit 110 into an estimation value of movement of the optical element.

The movement detection unit 106a is constituted by at least one of a processor and a logic circuit. The computer of the endoscope device 1a may read a program including commands defining the operations of the movement detection unit 106a and may execute the read program. In other words, the functions of the movement detection unit 106a may be realized by software.

The determination unit 110 determines whether or not a shock has occurred on the basis of the movement amount output from the movement detection unit 106a. Since the movement of the subject image is caused by movement of the distal end part 4a, the determination unit 110 can execute determination related to shock by using the movement of the subject image. When relative movement of the optical element to the imaging device 105 occurs, the determination unit 110 determines that the optical element moves from the first position or the second position. As an example in which the determination unit 110 determines that the optical element moves from the first position or the second position, an example in which the determination unit 110 determines whether or not a shock has occurred will be described.

Specifically, the determination unit 110 executes determination on the basis of the change in the movement amount on an image. For example, the determination unit 110 calculates the difference between a first movement amount and a second movement amount. Each of the first movement amount and the second movement amount is a movement amount detected by the determination unit 110 on the basis of two consecutive images. At least one of the two images for calculating the second movement amount is different from the two images for calculating the first movement amount.

The determination unit 110 compares the calculated difference with a threshold value, thus executing determination. If the difference exceeds the threshold value, the determination unit 110 determines that a shock has occurred. If the difference does not exceed the threshold value, the determination unit 110 determines that a shock has not occurred. For example, the amount of change in the movement amount corresponding to an acceleration when the shielding unit 210 of the optical path-switching unit 103 unintentionally moves is used as the threshold value. The relationship between the actual acceleration and the change in the movement amount of the subject image on an image is calculated on the basis of the magnification of optical systems and the size of pixels. The magnification indicates the size of the subject image projected on the imaging device 105. The movement amount of the subject image on an image changes in accordance with a subject distance. Therefore, the determination unit 110 may use the amount of change in the movement amount of the subject image at the maximum subject distance available in the image-matching technique as the threshold value in light of conditions of illumination light or the like. The determination unit 110 may execute simple distance measurement and may use the amount of change in the movement amount of the subject image on an image in accordance with the distance as the threshold value.

As described above, the acceleration when the shielding unit 210 of the optical path-switching unit 103 unintentionally moves is necessary for calculating the threshold value. The position of the shielding unit 210 is fixed by the holding force generated when the first permanent magnet 204 and the third permanent magnet 206 attract each other or the second permanent magnet 205 and the fourth permanent magnet 207 attract each other. By assuming a force exceeding the holding force, it is possible to calculate the threshold value. This threshold value is set mainly on the basis of the movement amount in the moving direction of the shielding unit 210 of the optical path-switching unit 103. The determination unit 110 sets the threshold value in light of other things in addition to the moving direction, thus executing more secure determination. As another method, the determination unit 110 may use only the movement amount of the subject image for the determination. The accuracy in a case in which the amount of change in the movement amount of the subject image is used is higher than that in a case in which the movement amount of the subject image is used.

When the operation mode is the measurement mode, the imaging device 105 generates the first image and the second image in accordance with the image-generation sequence of the measurement mode. Therefore, when optical paths are switched in accordance with the image-generation sequence, movement of the subject image occurs on the basis of the parallax. For example, in the example of the image-generation sequence of the measurement mode shown in FIG. 16, movement of the subject image occurs on the basis of the parallax between the first image “L” in the N-th frame and the second image “R” in the (N+1)-th frame. In addition, in the example of the image-generation sequence of the measurement mode shown in FIG. 16, movement of the subject image occurs between the second image “R” in the (N+1)-th frame and the first image “L” in the (N+2)-th frame. This movement needs to be distinguished from the movement of the subject image that occurs when images are switched due to a shock.

In the example of the image-generation sequence of the measurement mode shown in FIG. 17, since a shock occurs, the imaging device 105 generates the first image “L” instead of the second image “R” in the (N+1)-th frame. Therefore, the imaging device 105 continues to generate the first image “L” from the N-th to (N+2)-th frames. In the example of the image-generation sequence of the measurement mode shown in FIG. 17, movement of the subject image does not occur on the basis of the parallax between the first image “L” in the N-th frame and the first image “L” in the (N+1)-th frame. In addition, in the example of the image-generation sequence of the measurement mode shown in FIG. 17, movement of the subject image does not occur between the first image “L” in the (N+1)-th frame and the first image “L” in the (N+2)-th frame. If the operation mode is the measurement mode and the difference does not exceed the threshold value, the determination unit 110 determines that a shock has occurred. If the difference exceeds the threshold value, the determination unit 110 determines that a shock has not occurred. The threshold value in the measurement mode is the same as or different from the threshold value in the observation mode.

The operation of the endoscope device 1a is similar to that shown in FIG. 12 or FIG. 13. Two or more images are necessary for detecting a movement amount on an image. Therefore, Step S3a is not executed until the imaging device 105 generates two or more images. In other words, after Step S2 is executed, Step S51 is executed without executing Step 3a.

In the third embodiment, the endoscope device 1a can suppress the occurrence of a situation in which the imaging device 105 generates an image with the shielding unit 210 being disposed at an incorrect position as with the endoscope device 1 according to the second embodiment. The movement detection unit 106a detects movement of the optical element on the basis of images generated by the imaging device 105. The movement detection unit 106a does not need to be disposed in the distal end part 4a. Therefore, the distal end part 4a is miniaturized.

### Fourth Embodiment

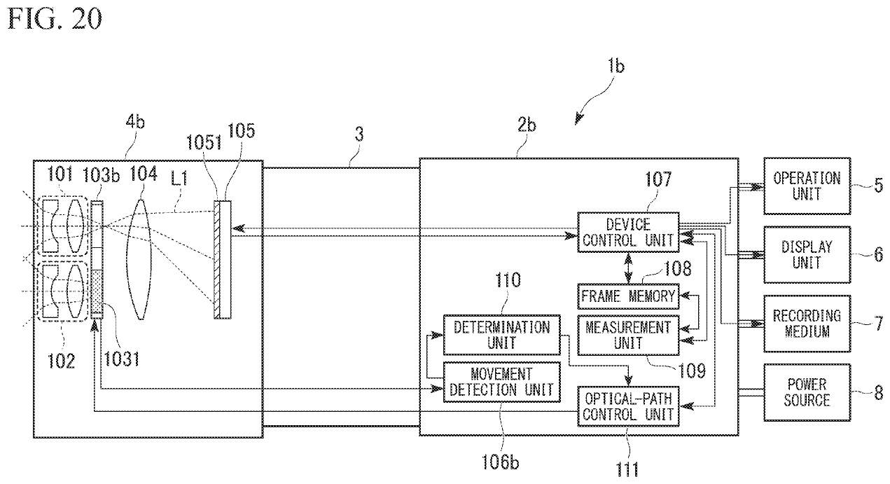

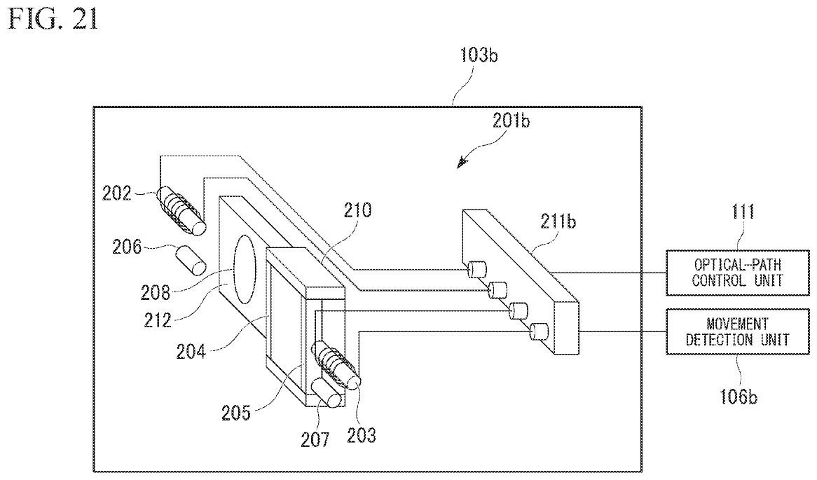

FIG. 20 shows a configuration of an endoscope device 1b according to a fourth embodiment of the present invention. The same configuration as that shown in FIG. 19 will not be described.

The main body unit 2a shown in FIG. 19 is changed to a main body unit 2b. The distal end part 4a shown in FIG. 19 is changed to a distal end part 4b. In the main body unit 2b, the movement detection unit 106a shown in FIG. 19 is changed to a movement detection unit 106b. In the distal end part 4b, the optical path-switching unit 103 shown in FIG. 19 is changed to an optical path-switching unit 103b. The optical path-switching unit 103b includes a shielding unit 1031 as with the optical path-switching unit 103 shown in FIG. 19.

FIG. 21 shows a configuration of a magnetic actuator 201b used as the optical path-switching unit 103b in the fourth embodiment. The same configuration as that shown in FIG. 7 and FIG. 8 will not be described. The current control unit 211 shown in FIG. 7 and FIG. 8 is changed to a current control unit 211b.

The current control unit 211b has a function of measuring the value related to the current in a circuit while the current is not applied to the first electromagnet 202 and the second electromagnet 203 in addition to the function of the current control unit 211 shown in FIG. 7 and FIG. 8. When the shielding unit 210 moves, the magnetic field in the first electromagnet 202 and the second electromagnet 203 changes. For example, when the shielding unit 210 moves from the first electromagnet 202 side to the second electromagnet 203 side, the magnetic field in the first electromagnet 202 reduces and the magnetic field in the second electromagnet 203 increases. In this way, in each of the first electromagnet 202 and the second electromagnet 203, the potential difference occurs due to electromagnetic induction and an induction current flows. The current control unit 211b measures the value of the induction current or the value of the potential difference due to electromagnetic induction and outputs the measured value to the movement detection unit 106b. The potential difference indicates the difference of the potential between two points in the first electromagnet 202 or the second electromagnet 203.

In the fourth embodiment, the movement detection unit 106b detects relative movement of the optical element to the imaging device 105. For example, the movement detection unit 106b detects movement of the optical element by detecting a current generated in the magnetic actuator 201b. Specifically, the movement detection unit 106b receives a measurement value of the induction current or the potential difference measured by the current control unit 211b and determines whether or not the shielding unit 210 has moved. In a case in which the current or the potential difference occurs in the first electromagnet 202 or the second electromagnet 203 while the current control unit 211b does not supply a current to the first electromagnet 202 and the second electromagnet 203, the current or the potential difference indicates that the magnetic field in the electromagnet has changed. In other words, the current or the potential difference indicates that the shielding unit 210 has unintentionally moved.

The movement detection unit 106b compares the measurement value with a threshold value. If the measurement value exceeds the threshold value, the movement detection unit 106b determines that the shielding unit 210 has moved. If the measurement value does not exceed the threshold value, the movement detection unit 106b determines that the shielding unit 210 has not moved. When the movement detection unit 106b determines that the shielding unit 210 has moved, the movement detection unit 106b outputs an abnormality detection signal to the determination unit 110.

When relative movement of the optical element to the imaging device 105 occurs, the determination unit 110 determines that the optical element moves from the first position or the second position. As an example in which the determination unit 110 determines that the optical element moves from the first position or the second position, an example in which the determination unit 110 determines whether or not a shock has occurred will be described. If the abnormality detection signal is output from the movement detection unit 106b, the determination unit 110 determines that a shock has occurred and outputs the shock occurrence signal to the optical-path control unit 111. If the abnormality detection signal is not output from the movement detection unit 106b, the determination unit 110 determines that a shock has not occurred.

The above-described function of the current control unit 211b may be defined as a function of a movement detection unit, and the movement detection unit 106b and the determination unit 110 may be integrated. The value of the current or the value of the potential difference measured by the current control unit 211b indicates movement of the shielding unit 210. The current control unit 211b outputs a measurement value to the determination unit 110. The determination unit 110 determines whether or not the shielding unit 210 has moved on the basis of the measurement value. The function of the determination unit 110 is the same as the above-described function of the movement detection unit 106b. If the shielding unit 210 has moved, the determination unit 110 determines that a shock has occurred and outputs the shock occurrence signal to the optical-path control unit 111. If the shielding unit 210 has not moved, the determination unit 110 determines that a shock has not occurred.

In the fourth embodiment, the endoscope device 1b can suppress the occurrence of a situation in which the imaging device 105 generates an image with the shielding unit 210 being disposed at an incorrect position as with the endoscope device 1 according to the second embodiment.

### Fifth Embodiment

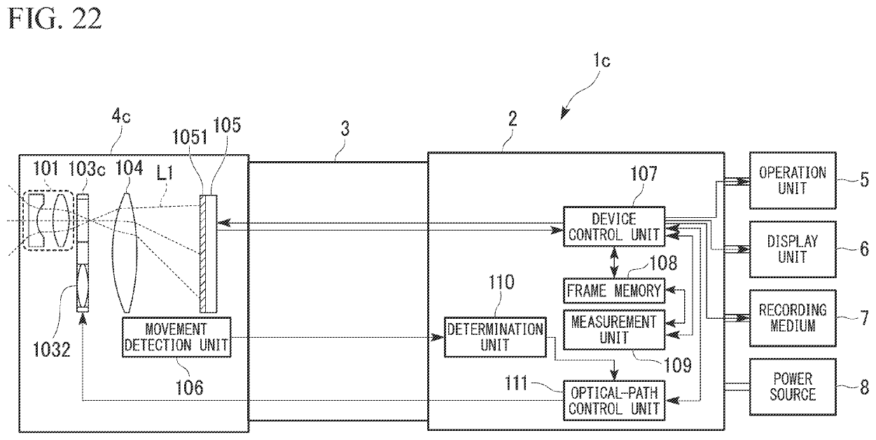

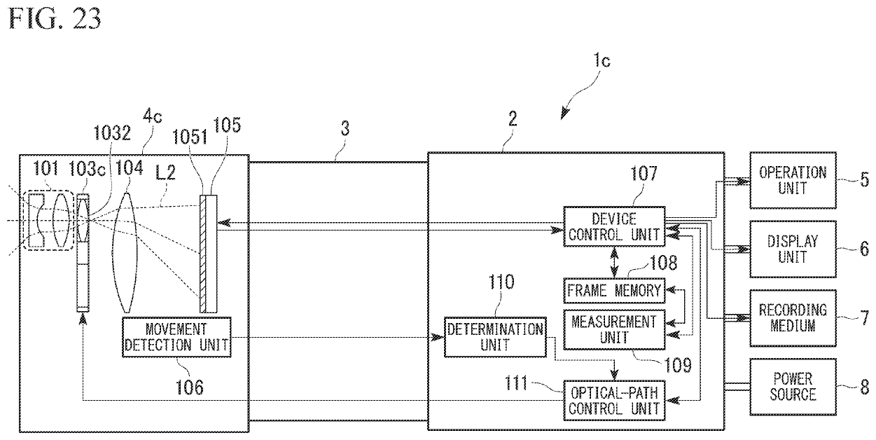

FIG. 22 and FIG. 23 show a configuration of an endoscope device 1c according to a fifth embodiment of the present invention. The same configuration as that shown in FIG. 5 and FIG. 6 will not be described.

The distal end part 4 shown in FIG. 5 and FIG. 6 is changed to a distal end part 4c. The distal end part 4c does not include the second optical system 102 shown in FIG. 5 and FIG. 6. In the distal end part 4c, the optical path-switching unit 103 shown in FIG. 5 and FIG. 6 is changed to an optical path-switching unit 103c.

The optical path-switching unit 103c includes a lens 1032 instead of the shielding unit 1031 shown in FIG. 5 and FIG. 6. The lens 1032 is disposed at any one of a first position on the optical path of the first optical system 101 and a second position away from the optical path of the first optical system 101.

In a case in which the lens 1032 is disposed at the second position as shown in FIG. 22, light passing through the first optical system 101 and the imaging optical system 104 is incident on the imaging region 1051 of the imaging device 105. The light passes through the first optical path L1. In this way, for example, a first optical image of which the focal point matches a near point is formed in the imaging region 1051. In a case in which the lens 1032 is disposed at the first position as shown in FIG. 23, light passing through the first optical system 101, the lens 1032, and the imaging optical system 104 is incident on the imaging region 1051 of the imaging device 105. The light passes through the second optical path L2. In this way, for example, a second optical image of which the focal point matches a far point is formed in the imaging region 1051. The optical path-switching unit 103c moves the lens 1032 from the first position to the second position or from the second position to the first position, thus switching between the first optical path L1 and the second optical path L2.

The optical-path control unit 111 controls the optical path-switching unit 103c, thus disposing the lens 1032 at any one of the first position and the second position. In this way, the optical-path control unit 111 controls the focus state of light incident on the imaging device 105.

In the fifth embodiment, the endoscope device 1c can suppress the occurrence of a situation in which the imaging device 105 generates an image with the lens 1032 being disposed at an incorrect position as with the endoscope device 1 according to the second embodiment. The endoscope device 1c can switch between the far point and the near point.

### Sixth Embodiment

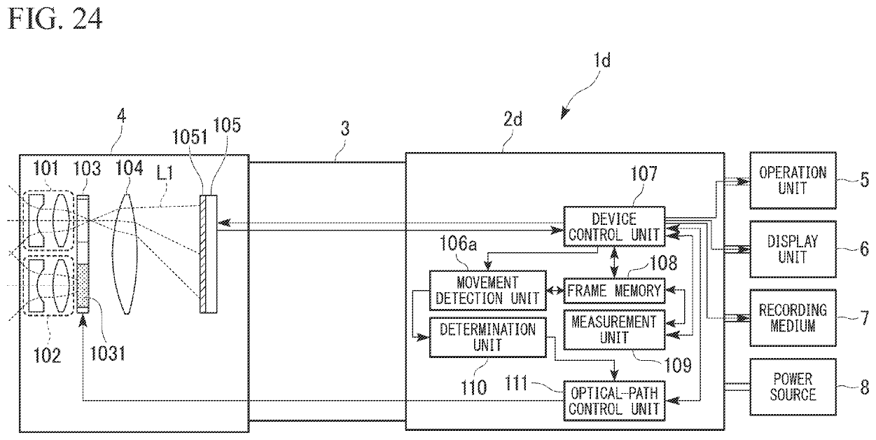

FIG. 24 shows a configuration of an endoscope device 1d according to a sixth embodiment of the present invention. The same configuration as that shown in FIG. 5 and FIG. 6 will not be described.

The main body unit 2 shown in FIG. 5 and FIG. 6 is changed to a main body unit 2d. In the main body unit 2d, the movement detection unit 106 shown in FIG. 5 and FIG. 6 is changed to a movement detection unit 106d.

The movement detection unit 106d reads the first image or the second image from the frame memory 108 and detects movement of the optical element on the basis of the first image or the second image. In the sixth embodiment, the movement detection unit 106d detects relative movement of the optical element to the imaging device 105. Specifically, the movement detection unit 106d detects a special state of an image. For example, the special state of an image is double images. Alternatively, the special state of an image is positional deviation of a subject image between two positions apart from each other in the vertical direction of the image. Alternatively, the special state of an image is the difference of focal points between two positions apart from each other in the vertical direction of the image.

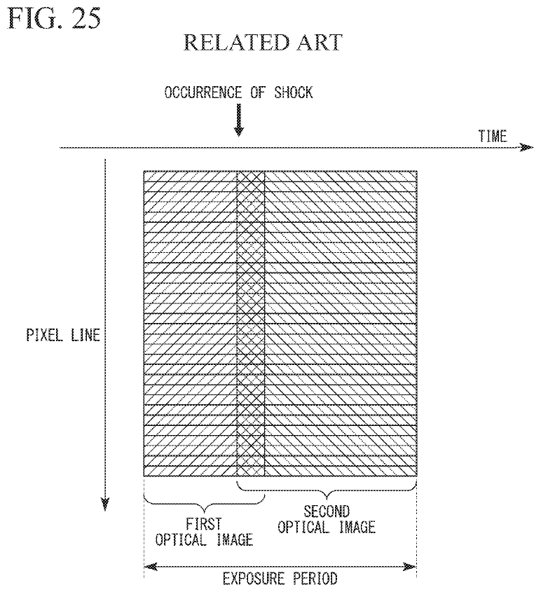

Double images occur in images generated by an imaging device using mainly a global shutter method. The global shutter method will be described with reference to FIG. 25. FIG. 25 shows an exposure period in an imaging device using the global shutter method.

The horizontal axis in FIG. 25 indicates time and the vertical axis in FIG. 25 indicates pixel lines of the imaging device. The imaging device includes a plurality of pixel lines. Each of the pixel lines includes a plurality of pixels.

In the global shutter method, an exposure period is simultaneously started in all the pixel lines and is simultaneously completed in all the pixel lines. In the example shown in FIG. 24, when the exposure period is started, the first optical path L1 is used as the imaging optical path. Therefore, the first optical image formed by light passing through the first optical path L1 is formed on all the pixel lines. A shock occurs in the exposure period, and the imaging optical path is changed from the first optical path L1 to the second optical path L2. After the imaging optical path is changed to the second optical path L2, the second optical image formed by light passing through the second optical path L2 is formed on all the pixel lines. While the imaging optical path is changed from the first optical path L1 to the second optical path L2, the first optical image and the second optical image are simultaneously formed on all the pixel lines.

Since the first optical image and the second optical image are simultaneously formed on the imaging device in the exposure period, the imaging device generates an image in which two subject images are seen. In other words, the imaging device generates an image in which double images are seen.

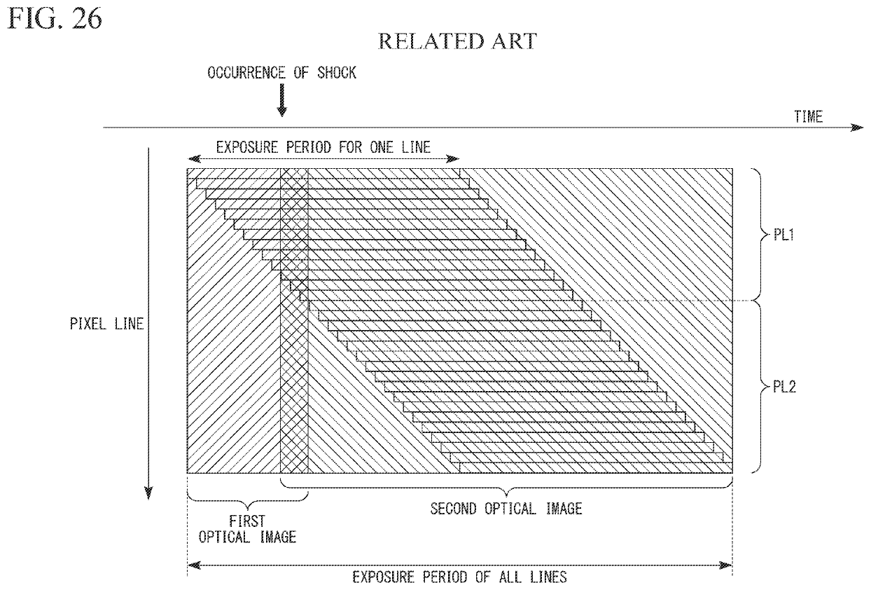

Double images occur also in an image generated by an imaging device using a rolling shutter method. The rolling shutter method will be described with reference to FIG. 26. FIG. 26 shows an exposure period in an imaging device using the rolling shutter method. As with FIG. 25, the horizontal axis in FIG. 26 indicates time and the vertical axis in FIG. 26 indicates pixel lines of the imaging device.

In the rolling shutter method, timings at which the exposure period is started are different from each other between the pixel lines, and timings at which the exposure period is completed are different from each other between the pixel lines. In the example shown in FIG. 26, the exposure period is sequentially started from the upper pixel line toward the lower pixel line. The start timing of the exposure period of each pixel line is shifted from the start timing of the exposure period of the pixel line adjacent to each pixel line by a predetermined amount of time.

In the example shown in FIG. 26, a shock occurs in the exposure period of some of the plurality of pixel lines disposed in the imaging device. In the pixel lines for which a shock occurs in the exposure period, the first optical image and the second optical image are simultaneously formed as with the example shown in FIG. 25. On the other hand, in the other pixel lines, the exposure period does not overlap the period in which the first optical image and the second optical image are simultaneously formed. In the example shown in FIG. 26, the first optical image and the second optical image are simultaneously formed in the exposure period of the upper pixel lines PL1. In the example shown in FIG. 26, only the second optical image is formed in the exposure period of the lower pixel lines PL2.

Since both the first optical image and the second optical image are formed in the exposure period of some of the plurality of pixel lines, the imaging device generates an image in which two subject images are seen. In other words, the imaging device generates an image in which double images are seen.

For example, the movement detection unit 106d uses a method of calculating autocorrelation of an image or a method of image-matching using part of an image, thus determining whether or not different regions of the image are similar to each other. In this way, the movement detection unit 106d determines whether or not double images occur.

The positional deviation of a subject image or the difference of focal points occurs in an image generated by an imaging device using mainly the rolling shutter method. As shown in FIG. 26, the first optical image and the second optical image are simultaneously formed in the exposure period of the pixel lines PL1, and only the second optical image is formed in the exposure period of the pixel lines PL2. Since the first optical image is formed in the pixel lines PL1 and the first optical image is not formed in the pixel lines PL2, the image shows discontinuity at the boundary between the pixel lines PL1 and the pixel lines PL2.

For example, the movement detection unit 106d detects a plurality of straight lines extending in the vertical direction of an image. The straight line included in the first optical image is seen in the image of the pixel lines PL1 but is not seen in the image of the pixel lines PL2. Therefore, the straight line included in the first optical image shows discontinuity at the boundary between the pixel lines PL1 and the pixel lines PL2. The movement detection unit 106d determines continuity of the plurality of straight lines extending in the vertical direction of the image. In a case in which all the straight lines show discontinuity at the same pixel line, the movement detection unit 106d determines that positional deviation of a subject image occurs. The movement detection unit 106d may use a similar method to that of detecting the positional deviation of a subject image in order to detect double images.

The distal end part 4 shown in FIG. 24 may be changed to the distal end part 4c shown in FIG. 22 and FIG. 23. The movement detection unit 106d determines continuity of a plurality of straight lines extending in the vertical direction of an image by using a similar method to that described above. As with the example shown in FIG. 26, all the straight lines show discontinuity at the same pixel line in a case in which both the first optical image and the second optical image are formed in the exposure period of some of the plurality of pixel lines. In a case in which the distal end part 4c is used, focal points are different from each other between the first optical image and the second optical image. Therefore, the movement detection unit 106d can detect the difference of focal points by determining continuity of straight lines.

When the special state of an image is detected, the movement detection unit 106d outputs an abnormality detection signal to the determination unit 110. When relative movement of the optical element to the imaging device 105 occurs, the determination unit 110 determines that the optical element moves from the first position or the second position. As an example in which the determination unit 110 determines that the optical element moves from the first position or the second position, an example in which the determination unit 110 determines whether or not a shock has occurred will be described. If the abnormality detection signal is output from the movement detection unit 106d, the determination unit 110 determines that a shock has occurred and outputs the shock occurrence signal to the optical-path control unit 111. If the abnormality detection signal is not output from the movement detection unit 106d, the determination unit 110 determines that a shock has not occurred.

In the sixth embodiment, the endoscope device 1d can suppress the occurrence of a situation in which the imaging device 105 generates an image with the shielding unit 210 being disposed at an incorrect position as with the endoscope device 1 according to the second embodiment.

### Seventh Embodiment

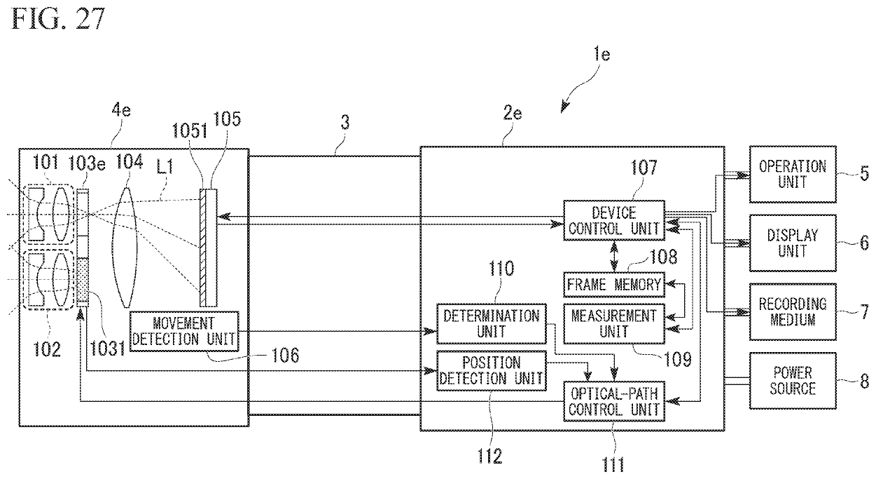

FIG. 27 shows a configuration of an endoscope device 1e according to a seventh embodiment of the present invention. The same configuration as that shown in FIG. 5 and FIG. 6 will not be described.

The main body unit 2 shown in FIG. 5 and FIG. 6 is changed to a main body unit 2e. The distal end part 4 shown in FIG. 5 and FIG. 6 is changed to a distal end part 4e. The main body unit 2e includes a position detection unit 112 in addition to the configuration FIG. 5 and FIG. 6. In the distal end part 4e, the optical path-switching unit 103 shown in FIG. 5 and FIG. 6 is changed to an optical path-switching unit 103e. The optical path-switching unit 103e includes the shielding unit 1031 as with the optical path-switching unit 103 shown in FIG. 5 and FIG. 6. The position detection unit 112 detects the position of the shielding unit 1031.

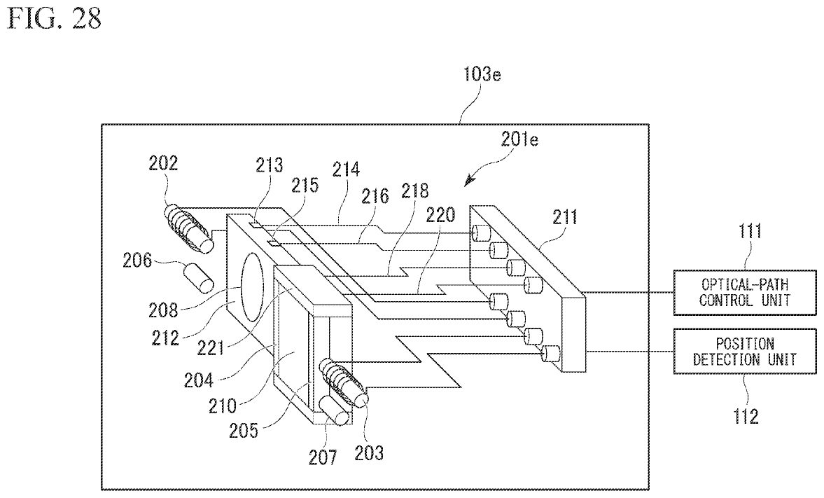

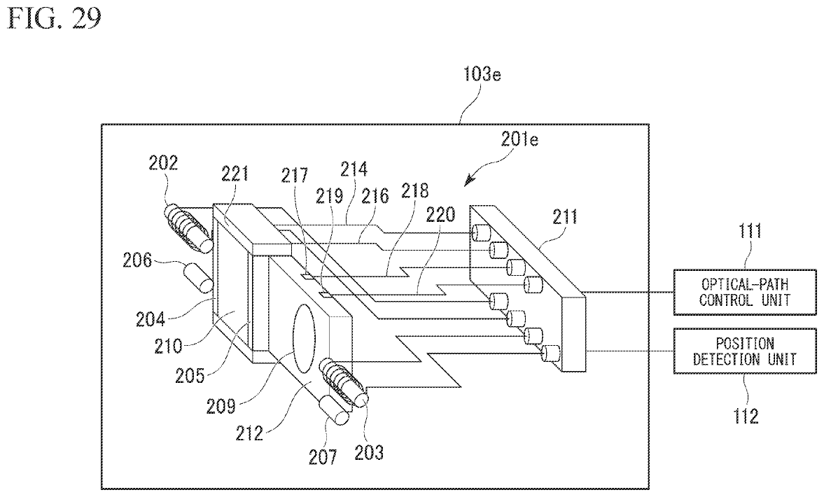

FIG. 28 and FIG. 29 show a configuration of a magnetic actuator 201e used as the optical path-switching unit 103e in the seventh embodiment. The same configuration as that shown in FIG. 7 and FIG. 8 will not be described. FIG. 28 shows a state in which the shielding unit 210 shields the second opening 209. FIG. 29 shows a state in which the shielding unit 210 shields the first opening 208. The magnetic actuator 201e includes a terminal 213, a terminal 215, a terminal 217, a terminal 219, a signal line 214, a signal line 216, a signal line 218, a signal line 220, and a switch 221 in addition to the configuration shown in FIG. 7 and FIG. 8.

The terminal 213, the terminal 215, the terminal 217, and the terminal 219 are disposed on the surface of the light selection unit 212. The signal line 214 electrically connects the terminal 213 and the current control unit 211 together. The signal line 216 electrically connects the terminal 215 and the current control unit 211 together. The signal line 218 electrically connects the terminal 217 and the current control unit 211 together. The signal line 220 electrically connects the terminal 219 and the current control unit 211 together.

The switch 221 constituted of conductor is attached to the shielding unit 210. As shown in FIG. 28, when the shielding unit 210 is disposed at the second position so as to shield the second opening 209, the terminal 217 and the terminal 219 come into contact with the switch 221. At this time, the current output from the current control unit 211 flows in a second circuit including the terminal 217, the terminal 219, the signal line 218, the signal line 220, and the switch 221. As shown in FIG. 29, when the shielding unit 210 is disposed at the first position so as to shield the first opening 208, the terminal 213 and the terminal 215 come into contact with the switch 221. At this time, the current output from the current control unit 211 flows in a first circuit including the terminal 213, the terminal 215, the signal line 214, the signal line 216, and the switch 221.

Accordingly, when the shielding unit 210 is disposed at the second position in order to use the first optical path L1 as the imaging optical path, the current flows in the second circuit but does not flow in the first circuit. When the shielding unit 210 is disposed at the first position in order to use the second optical path L2 as the imaging optical path, the current flows in the first circuit but does not flow in the second circuit. The current control unit 211 determines a circuit in which the current flows among the first circuit and the second circuit. The current control unit 211 outputs information of the circuit in which the current flows to the position detection unit 112.

The first circuit and the second circuit may have respective resistance values and may be connected in parallel with each other. When a predetermined voltage is applied to the first circuit and the second circuit, the amount of current flowing in the first circuit and the amount of current flowing in the second circuit are different from each other. The current control unit 211 may measure the amount of current output from the entire first circuit and second circuit and may output the measured value to the position detection unit 112. The value is different in accordance with the circuit in which the current flows.

When the information of the circuit in which the current flows is output from the current control unit 211, the position detection unit 112 determines the position of the shielding unit 210 on the basis of the information. When the information of the first circuit is output from the current control unit 211, the position detection unit 112 determines that the shielding unit 210 is disposed at the first position. When the information of the second circuit is output from the current control unit 211, the position detection unit 112 determines that the shielding unit 210 is disposed at the second position.

When the measured value of the current is output from the current control unit 211, the position detection unit 112 determines the position of the shielding unit 210 on the basis of the measured value. For example, when a predetermined voltage is applied to the first circuit and the second circuit, the amount of current flowing in the first circuit is greater than the amount of current flowing in the second circuit. The position detection unit 112 compares the measured value with a threshold value. The threshold value is a value between the measured value of the current flowing in the first circuit and the measured value of the current flowing in the second circuit. When the measured value is greater than the threshold value, the position detection unit 112 determines that the current flows in the first circuit, that is, the shielding unit 210 is disposed at the first position. When the measured value is less than the threshold value, the position detection unit 112 determines that the current flows in the second circuit, that is, the shielding unit 210 is disposed at the second position.

The position detection unit 112 is constituted by at least one of a processor and a logic circuit. The computer of the endoscope device 1e may read a program including commands defining the operations of the position detection unit 112 and may execute the read program. In other words, the functions of the position detection unit 112 may be realized by software.

The position detection unit 112 outputs position information indicating the position of the shielding unit 210 to the optical-path control unit 111.

Positions at which the shielding unit 210 is disposed and the number of times the imaging device 105 generates each of the first image and the second image are set in advance as the sequence information. When the determination unit 110 determines that a shock has occurred, the optical-path control unit 111 confirms the position of the shielding unit 210 on the basis of the position information output from the position detection unit 112. When the confirmed position is the same as a predetermined position necessary for the imaging device 105 to generate the first image or the second image, the device control unit 107 does not execute the disposition control and causes the imaging device 105 to continue imaging. The predetermined position is any one of the first position and the second position. When the operation mode is the measurement mode, the device control unit 107 causes the imaging device 105 to continue stereo imaging.

When the operation mode is the measurement mode, the shielding unit 210 is first disposed at an initial position in the image-generation sequence. The initial position is any one of the first position and the second position. When the confirmed position is different from the predetermined position necessary for the imaging device 105 to generate the first image or the second image, the optical-path control unit 111 executes the disposition control and causes the imaging device 105 to start stereo imaging.

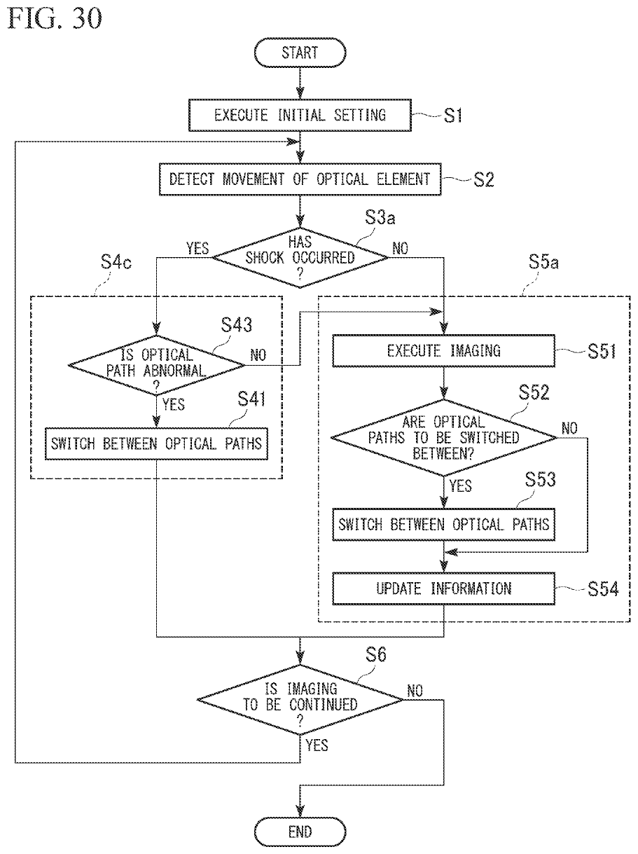

An operation of the endoscope device 1e in the observation mode or the measurement mode will be described with reference to FIG. 30. FIG. 30 shows a procedure of processing executed by the endoscope device 1e for switching between optical paths and continuing the image-generation sequence when a shock occurs. In the processing shown in FIG. 30, imaging is resumed in order to execute the rest of the image-generation sequence after a shock occurs. The same processing as that shown in FIG. 12 will not be described. As an example in which the determination unit 110 determines that the optical element moves from the first position or the second position, an example in which the determination unit 110 determines whether or not a shock has occurred will be described.

When the determination unit 110 determines that a shock has occurred in Step S3a, the determination unit 110 outputs the shock occurrence signal to the optical-path control unit 111. The optical-path control unit 111 executes the restoration control (Step S4c). In the restoration control, the following Step S43 is executed. After Step S43, Step S41 is executed in the restoration control, or the normal control (Step S5a) is executed.

The optical-path control unit 111 confirms the position of the shielding unit 210 on the basis of the position information output from the position detection unit 112. The optical-path control unit 111 determines whether or not the optical path is abnormal on the basis of the sequence information (Step S43). When the position of the shielding unit 210 indicated by the sequence information and the position indicated by the position information are different from each other, the optical-path control unit 111 determines that the optical path is abnormal. When the position of the shielding unit 210 indicated by the sequence information and the position indicated by the position information are the same, the optical-path control unit 111 determines that the optical path is not abnormal.

When the optical-path control unit 111 determines that the optical path is abnormal in Step S43, Step S41 is executed. Since the optical-path control unit 111 switches between optical paths, the imaging optical path is changed to a correct optical path in the image-generation sequence.

When the optical-path control unit 111 determines that the optical path is not abnormal in Step S43, Step S51 is executed in the normal control. The imaging device 105 executes imaging in accordance with the image-generation sequence in Step S51.

The processing shown in FIG. 13 may include above-described Step S43.

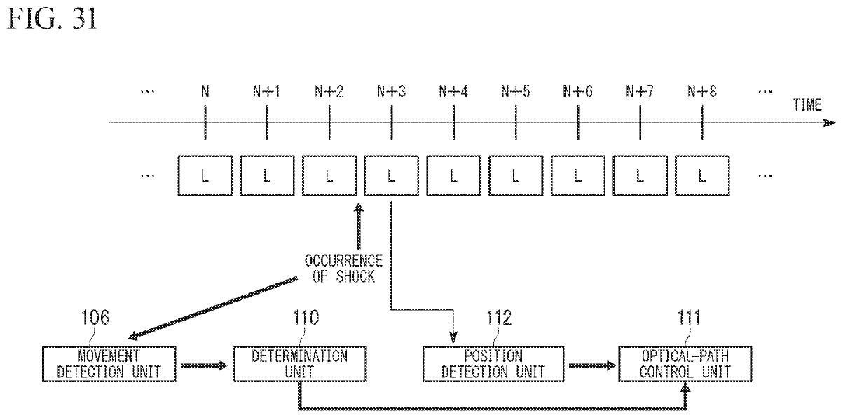

FIGS. 31 to 34 show specific examples of the image-generation sequence. In FIGS. 31 to 34, the horizontal axis indicates time. In FIGS. 31 to 34, the first image is shown as “L” and the second image is shown as “R.” In FIGS. 31 to 34, frame numbers are shown on the upper side of the axis indicating passage of time. The character N included in the frame number indicates an integer of one or more.

FIG. 31 shows an example of the image-generation sequence in a case in which a shock occurs in the observation mode. In the example shown in FIG. 31, the shielding unit 210 does not move and the position of the shielding unit 210 is appropriate after a shock occurs.

The imaging device 105 generates the first image “L” from the N-th frame to the (N+2)-th frame. The movement detection unit 106 detects movement of the distal end part 4e in each frame. The determination unit 110 detects a shock after transfer of image data is completed in the (N+2)-th frame. The position detection unit 112 detects the position of the shielding unit 210 in the (N+3)-th frame. Since the shielding unit 210 does not move, the optical-path control unit 111 confirms that the position of the shielding unit 210 is the same as the position indicated by the sequence information. Therefore, the optical-path control unit 111 does not execute the switching control.

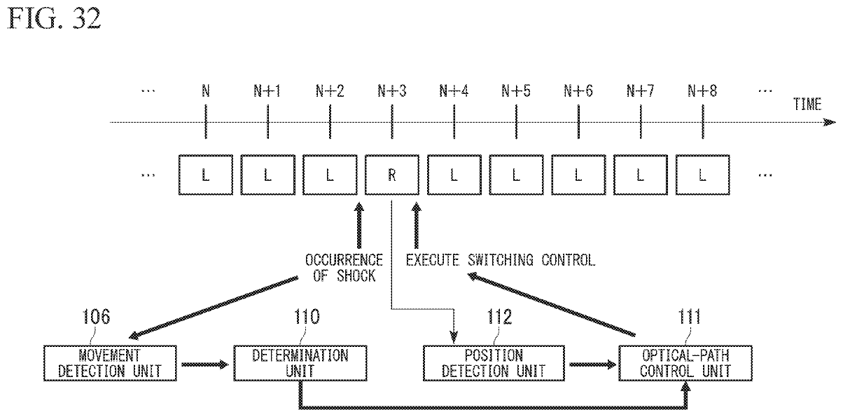

FIG. 32 shows an example of the image-generation sequence in a case in which a shock occurs in the observation mode. In the example shown in FIG. 32, the shielding unit 210 moves after a shock occurs.

Imaging is started as with the example shown in FIG. 31. The movement detection unit 106 detects movement of the distal end part 4e in each frame. A shock occurs at the same timing as that shown in FIG. 31. In the example shown in FIG. 32, the determination unit 110 detects a shock after transfer of image data is completed in the (N+2)-th frame. The position detection unit 112 detects the position of the shielding unit 210 in the (N+3)-th frame. Since the shielding unit 210 moves, the optical-path control unit 111 confirms that the position of the shielding unit 210 is different from the position indicated by the sequence information. Therefore, the optical-path control unit 111 executes the switching control.

The optical-path control unit 111 switches between optical paths after transfer of image data is completed in the (N+3)-th frame. The imaging device 105 generates the second image “R” in the (N+3)-th frame and generates the first image “L” in the (N+4)-th frame and each of the following frames.

Since the optical-path control unit 111 switches between optical paths, the imaging device 105 can generate appropriate images in the (N+4)-th frame and each of the following frames. Even when a different image from the scheduled image is generated, the endoscope device 1e can change the imaging optical path to a predetermined optical path necessary for observation and can continue to generate appropriate images.

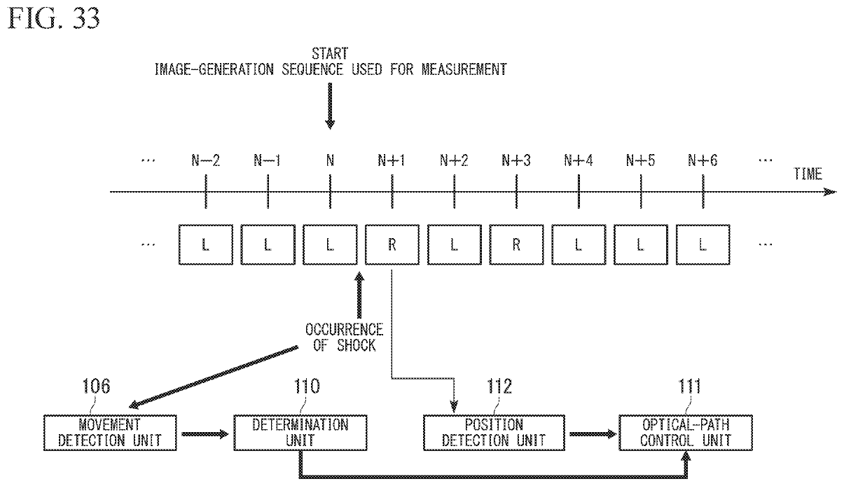

FIG. 33 shows an example of the image-generation sequence in a case in which a shock occurs in the measurement mode. In the example shown in FIG. 33, the shielding unit 210 does not move and the position of the shielding unit 210 is appropriate after a shock occurs.

The imaging device 105 generates the first image “L” in each of the (N−2)-th frame and the (N−1)-th frame. Thereafter, the operation mode is changed to the measurement mode, and the image-generation sequence used for measurement is started in the N-th frame. The imaging device 105 generates the first image “L” in the N-th frame. The movement detection unit 106 detects movement of the distal end part 4e in each frame. The determination unit 110 detects a shock after transfer of image data is completed in the N-th frame. The position detection unit 112 detects the position of the shielding unit 210 in the (N+1)-th frame. Since the shielding unit 210 does not move, the optical-path control unit 111 confirms that the position of the shielding unit 210 is the same as the position indicated by the sequence information. Therefore, the optical-path control unit 111 does not execute the switching control.

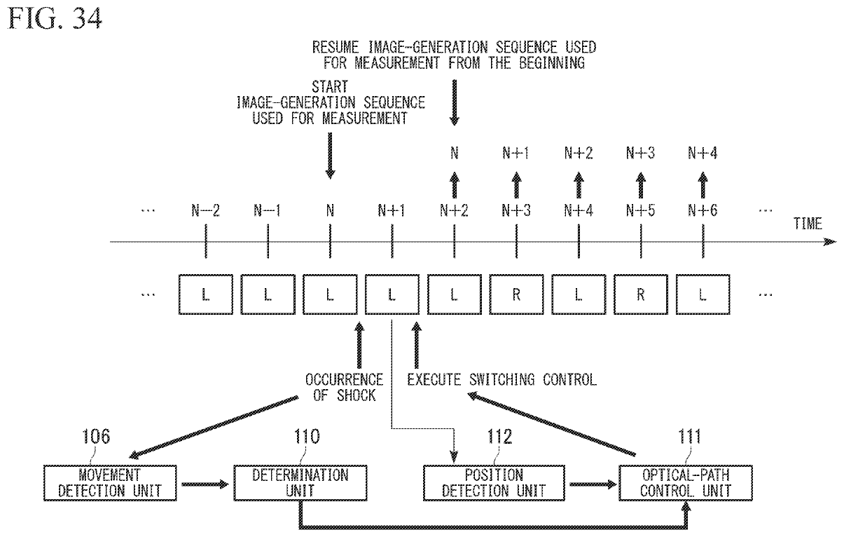

FIG. 34 shows an example of the image-generation sequence in a case in which a shock occurs in the measurement mode. In the example shown in FIG. 34, the shielding unit 210 moves after a shock occurs.

The imaging device 105 generates the first image “L” in each of the (N−2)-th frame and the (N−1)-th frame, and the image-generation sequence used for measurement is started in the N-th frame as with the example shown in FIG. 33. The imaging device 105 generates the first image “L” in the N-th frame. The movement detection unit 106 detects movement of the distal end part 4e in each frame. A shock occurs at the same timing as that shown in FIG. 33. In the example shown in FIG. 34, the determination unit 110 detects a shock after transfer of image data is completed in the N-th frame. The position detection unit 112 detects the position of the shielding unit 210 in the (N+1)-th frame. Since the shielding unit 210 moves, the optical-path control unit 111 confirms that the position of the shielding unit 210 is different from the position indicated by the sequence information. Therefore, the optical-path control unit 111 executes the switching control.

The optical-path control unit 111 sets the imaging optical path in the (N+2)-th frame to the same imaging optical path as that in the N-th frame. The image-generation sequence is repeated from the (N+2)-th frame to the (N+6)-th frame. The imaging device 105 generates the first image “L” or the second image “R” in accordance with the image-generation sequence in each of the (N+2)-th to the (N+6)-th frames.

The optical-path control unit 111 sets the imaging optical path to the same imaging optical path as that first set in the image-generation sequence, and the image-generation sequence is resumed from the beginning. Therefore, the endoscope device 1e can generate images necessary in the measurement unit 109. The measurement unit 109 can correctly execute measurement.

In the endoscope device 1a shown in FIG. 19, the optical path-switching unit 103 may include a similar configuration to the magnetic actuator 201e, and the main body unit 2a may include the position detection unit 112.

In the endoscope device 1b shown in FIG. 20, the optical path-switching unit 103b may include a similar configuration to the magnetic actuator 201e except for the current control unit 211b, and the main body unit 2b may include the position detection unit 112.

In the endoscope device 1c shown in FIG. 22 and FIG. 23, the optical path-switching unit 103c may include a similar configuration to the magnetic actuator 201e except for the lens 1032, and the main body unit 2 may include the position detection unit 112.

In the endoscope device 1d shown in FIG. 24, the optical path-switching unit 103 may include a similar configuration to the magnetic actuator 201e, and the main body unit 2d may include the position detection unit 112.

In the seventh embodiment, the endoscope device 1e can suppress the occurrence of a situation in which the imaging device 105 generates an image with the shielding unit 210 being disposed at an incorrect position as with the endoscope device 1 according to the second embodiment. When a shock has occurred, the optical-path control unit 111 confirms the position of the shielding unit 210. When the position of the shielding unit 210 is different from a correct position indicated by the sequence information, the optical-path control unit 111 switches between optical paths. When the position of the shielding unit 210 is the same as the correct position indicated by the sequence information, the optical-path control unit 111 does not switch between optical paths. Accordingly, the optical-path control unit 111 can correctly execute switching of optical paths.

In the above-described example, when the position of the shielding unit 210 is different from the correct position, the optical-path control unit 111 switches between optical paths. When the position of the shielding unit 210 is different from the correct position, the endoscope device 1e may start processing in accordance with the position of the shielding unit 210.

### Eighth Embodiment

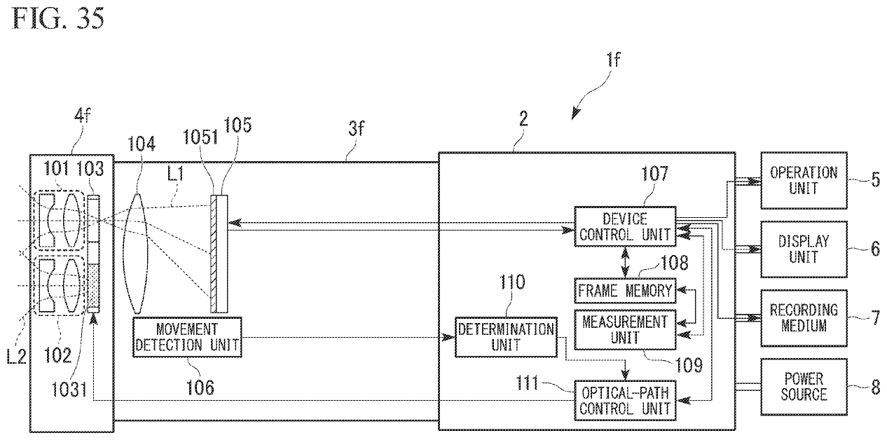

FIG. 35 shows a configuration of an endoscope device if according to an eighth embodiment of the present invention. The same configuration as that shown in FIG. 5 and FIG. 6 will not be described.

The insertion unit 3 shown in FIG. 5 and FIG. 6 is changed to an insertion unit 3f. The distal end part 4 shown in FIG. 5 and FIG. 6 is changed to a distal end part 4f. The configuration of the distal end part 4 shown in FIG. 5 and FIG. 6 is disposed in the insertion unit 3f and the distal end part 4f. The insertion unit 3f includes the imaging optical system 104, the imaging device 105, and the movement detection unit 106. The imaging optical system 104, the imaging device 105, and the movement detection unit 106 are disposed in the distal end of the insertion unit 3f. The distal end part 4f includes the first optical system 101, the second optical system 102, and the optical path-switching unit 103. The imaging optical system 104 may be disposed in the distal end part 4f. The first optical system 101, the second optical system 102, and the optical path-switching unit 103 may be disposed in the insertion unit 3f without the distal end part 4f being disposed.

In the eighth embodiment, the endoscope device 1f can suppress the occurrence of a situation in which the imaging device 105 generates an image with the shielding unit 210 being disposed at an incorrect position as with the endoscope device 1 according to the second embodiment.

While preferred embodiments of the invention have been described and shown above, it should be understood that these are examples of the invention and are not to be considered as limiting. Additions, omissions, substitutions, and other modifications can be made without departing from the spirit or scope of the present invention.

Accordingly, the invention is not to be considered as being limited by the foregoing description, and is only limited by the scope of the appended claims.

What is claimed is:

1. An endoscope device, comprising:
an endoscope comprising, in a tip end thereof,
an imaging device;
a first optical system configured to lead light from a subject to the imaging device; and
an optical element that is disposed at any one of a first position on an optical path of the first optical system and a second position away from the optical path of the first optical system and is configured to switch between states of light incident on the imaging device;
a detector configured to detect movement of the optical element; and
a processor configured to:
determine whether or not the optical element moves from the first position or the second position on the basis of the movement;
execute disposition control of disposing the optical element at the first position or the second position; and
execute the disposition control in a predetermined state, the predetermined state being a state in which the processor determines that the optical element moves from the first position or the second position after the disposition control is executed and before the disposition control is executed next.

2. The endoscope device according to claim 1,
wherein the processor is configured to determine whether or not the optical element moves from the first position or the second position by determining whether or not a force exceeding a predetermined amount is added to the optical element.

3. The endoscope device according to claim 1,
wherein the processor is configured to determine that the optical element moves from the first position or the second position when relative movement of the optical element to the imaging device occurs.

4. The endoscope device according to claim 1,
wherein the endoscope comprises a second optical system that is different from the first optical system and is configured to lead light from the subject to the imaging device,
the optical element is a shutter that cuts off light,
the second position is on an optical path of the second optical system,
the imaging device is configured to generate a first image on the basis of a first optical image formed by light passing through the first optical system when the optical element is disposed at the second position,
the imaging device is configured to generate a second image on the basis of a second optical image formed by light passing through the second optical system when the optical element is disposed at the first position, and
the imaging device is configured to execute stereo imaging for generating the first image one or more times and generating the second image one or more times.

5. The endoscope device according to claim 4,
wherein the processor is configured to:
dispose the optical element at an initial position that is set in advance among the first position and the second position by executing the disposition control and causing the imaging device to start the stereo imaging; and
after the imaging device starts the stereo imaging, execute the disposition control of disposing the optical element at any one of the first position and the second position in the predetermined state and cause the imaging device to continue the stereo imaging

6. The endoscope device according to claim 5,
wherein, after the imaging device starts the stereo imaging, the processor is configured to execute the disposition control of disposing the optical element at the initial position in the predetermined state so as to cause the imaging device to continue the stereo imaging

7. The endoscope device according to claim 4,
wherein the processor is configured to:
confirm a position of the optical element in the predetermined state;
skip executing the disposition control and cause the imaging device to continue the stereo imaging when the confirmed position is the same as a predetermined position necessary for the imaging device to next generate the first image or the second image; and
execute the disposition control and cause the imaging device to continue the stereo imaging when the confirmed position is different from the predetermined position,
wherein the predetermined position is any one of the first position and the second position.

8. The endoscope device according to claim 1,
wherein, in the predetermined state, by executing the disposition control, the processor is configured to dispose the optical element at any one of:
an initial position that is set in advance among the first position and the second position;
a position at which the optical element is disposed through the disposition control executed immediately before the predetermined state occurs; and
a position at which the optical element is scheduled to be disposed through the disposition control to be executed next.

9. The endoscope device according to claim 1,
wherein the optical element is a lens, and
the processor is configured to dispose the optical element at any one of the first position and the second position so as to control a focus state of light incident on the imaging device.

10. The endoscope device according to claim 1, further comprising a magnetic actuator configured to move the optical element from the first position to the second position or from the second position to the first position,
wherein the detector is configured to detect the movement by detecting a current generated in the magnetic actuator.

11. The endoscope device according to claim 1,
wherein the detector is an acceleration sensor or a gyro sensor.

12. The endoscope device according to claim 1,
wherein the detector is configured to detect the movement on the basis of an image generated by the imaging device.

13. The endoscope device according to claim 2, further comprising a holding mechanism configured to hold the optical element at the first position or the second position by using a predetermined amount of force.

14. A method of operating an endoscope device,
wherein the endoscope device includes:
an endoscope including, in a tip end thereof,
an imaging device;
a first optical system configured to lead light from a subject to the imaging device; and
an optical element that is disposed at any one of a first position on an optical path of the first optical system and a second position away from the optical path of the first optical system and is configured to switch between states of light incident on the imaging device,
the method comprising:
a detection step of causing a detector to detect movement of the optical element;
a determination step of causing a processor to determine whether or not the optical element moves from the first position or the second position on the basis of the movement;
a first control step of causing the processor to execute disposition control of disposing the optical element at the first position or the second position; and
a second control step of causing the processor to execute the disposition control in a predetermined state, the predetermined state being a state in which the processor determines that the optical element moves from the first position or the second position after the disposition control is executed and before the disposition control is executed next.

15. A non-transitory computer-readable recording medium saving a program executed by a computer of an endoscope device,
wherein the endoscope device includes:
an endoscope including, in a tip end thereof,
an imaging device;
a first optical system configured to lead light from a subject to the imaging device; and
an optical element that is disposed at any one of a first position on an optical path of the first optical system and a second position away from the optical path of the first optical system and is configured to switch between states of light incident on the imaging device,
the computer executes:
a detection step of detecting movement of the optical element;
a determination step of determining whether or not the optical element moves from the first position or the second position on the basis of the movement;
a first control step of executing disposition control of disposing the optical element at the first position or the second position; and
a second control step of executing the disposition control in a predetermined state, the predetermined state being a state in which it is determined, in the determination step, that the optical element moves from the first position or the second position after the disposition control is executed and before the disposition control is executed next.
